(19) United States
(12) Patent Application Publication
(10) Pub. No.: US 2024/0358921 A1
(43) Pub. Date: Oct. 31, 2024

(54) DEVICES, SYSTEMS, AND METHODS FOR COMBINING AND/OR DELIVERING INJECTABLE MATERIALS

(71) Applicants: BOSTON SCIENTIFIC SCIMED, INC., MAPLE GROVE, MN (US); BOSTON SCIENTIFIC MEDICAL DEVICE LIMITED, Galway (IE)

(72) Inventors: Nitesh Ghananil Baviskar, Kalyan West (IN); Benjamin Cleveland, Bellingham, MA (US); Sanjay Kumar Chandra, Pune (IN); Katie Knowles, Providence, RI (US); Marshall O'Hearn, Davenport, FL (US)

(73) Assignees: BOSTON SCIENTIFIC SCIMED, INC., MAPLE GROVE, MN (US); BOSTON SCIENTIFIC MEDICAL DEVICE LIMITED, Galway (IE)

(21) Appl. No.: 18/646,347

(22) Filed: Apr. 25, 2024

Related U.S. Application Data
(60) Provisional application No. 63/462,113, filed on Apr. 26, 2023.

(51) Int. Cl. A61M 5/19; A61M 5/31; A61M 5/315; A61M 5/32

(52) U.S. Cl. CPC A61M 5/19; A61M 5/3129; A61M 5/31511; A61M 5/3202

(57) ABSTRACT

A multi-chamber device having a barrel with a first chamber and a second chamber, and a plunger assembly with a first plunger rod advanceable with respect to the first chamber and a second plunger rod advanceable with respect to the second chamber. The ends of the first and second plunger rods are independently advanceable to advance different amounts of material from the first and second chamber as the plunger assembly is advanced with respect to the barrel. The multi-chamber device may be part of a multi-chamber system including an injection system. The multi-chamber device and the injection system may include engaging elements facilitating ready coupling and decoupling of the multi-chamber device and the injection system.

### CROSS-REFERENCE TO RELATED APPLICATIONS

This application claims the benefit of priority under 35 U.S.C. § 119 to U.S. Provisional Application No. 63/462,113, filed Apr. 26, 2023, the entire disclosure of which is hereby incorporated by reference herein for all purposes.

### FIELD

The present disclosure relates generally to the field of devices for delivering injectable compositions to a patient, and associated systems and methods. More particularly, the present disclosure relates to devices for combining components of injectable compositions, and associated systems and methods.

Various forms of cancer and other medical conditions are treated by local application of radiation therapy. However, various risks may accompany radiation therapy. Since the conception of conformal radiotherapy, physicians have paid attention to the radiation dose delivered to the target and surrounding tissues. Investigators have been able to correlate side effects to the amount of tissue receiving a certain radiation dose. And yet, time, distance, and shielding affect the dose that is delivered. The less time an area is exposed to radiation, the less dose is delivered. The greater the distance from the radiation, the less dose is delivered. Filler materials may be injected into a treatment area to provide a shield to tissue surrounding the target of the radiation therapy. For instance, numerous men are diagnosed with prostate cancer each year. Traditionally, treatment options include interstitial implant therapy, surgery, and external beam radiotherapy. While the best treatment is still debatable, side effects of treating prostate cancer have become less toxic with implant therapy and radiotherapy.

Various systems provide filler material to treatment sites to decrease the radiation dose to tissue surrounding radiation target sites (e.g., to shield the rectum during radiotherapy for prostate cancer). Such filler materials are often reactive, and therefore are generally combined/mixed immediately prior to or even during delivery to the patient. Various systems are known for combining/mixing (e.g., in vitro) filler materials injected into radiation treatment areas. However, most such systems include numerous subcomponents, are complex to assemble, and are susceptible to filler mixing errors prior to delivery within a patient at a treatment site. Various challenges posed by such mixing systems may result in errors and mishaps which lead unnecessarily increased procedure time, and increased procedure costs. Solutions to these and other issues presented by combining and delivering injectable materials would be welcome in the art.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a multi-chamber device includes a barrel defining a first chamber and a second chamber fluidly isolated from each other; and a plunger assembly having a first plunger rod with a distal end extendable with respect to the first chamber, and a second plunger rod with a distal end extendable with respect to the second chamber. In some aspects, a portion of the first plunger rod and a portion of the second plunger rod move together with respect to the barrel; and a portion of the first plunger rod is independently movable with respect to a portion of the second plunger rod to advance the distal end of first plunger rod with respect to the barrel a first extent and to advance the distal end of the second plunger rod with respect to the barrel a second extent different from the first extent.

In some aspects, the portion of the first plunger rod and the portion of the second plunger rod which are independently movable are the distal end of the first plunger rod and the distal end of the second plunger rod. In some aspects, a portion of the first plunger rod is coupled with a portion of the second plunger rod so that the portion of the first plunger rod and the portion of the second plunger rod move together. In some aspects, the portion of the first plunger rod and the portion of the second plunger rod which move together are a proximal end of the first plunger rod and a proximal end of the second plunger rod. In some aspects, the second plunger rod comprises a proximal plunger rod and a distal plunger rod axially movable with respect to each other; and the proximal plunger rod and the first plunger rod are coupled together to move together. In some aspects, the proximal plunger rod and the distal plunger rod are selectively movable into a position in which the proximal plunger rod and the distal plunger rod are engaged to move together. In some aspects, the multi-chamber device further includes a retainer coupled to the barrel to retain a portion of the second plunger rod in a positioned engaged with the retainer. In some aspects, the retainer engages the distal plunger rod in a distally advanced position with respect to the second chamber of the barrel. In some aspects, the proximal plunger rod and the distal plunger rod are selectively movable into a position in which the proximal plunger rod and the distal plunger rod are engaged to move together upon proximally retracting the proximal plunger rod with respect to the distal plunger rod.

In some aspects, the second plunger rod comprises a proximal plunger rod and a distal plunger rod axially movable with respect to each other; and the proximal plunger rod and the first plunger rod are coupled together to move together. In some aspects, the proximal plunger rod and the distal plunger rod are selectively movable into a position in which the proximal plunger rod and the distal plunger rod are engaged to move together.

In some aspects, the multi-chamber device further includes a retainer coupled to the barrel to retain a portion of the second plunger rod in a positioned engaged with the retainer.

In accordance with various principles of the present disclosure, a multi-chamber system includes a multi-chamber device and a vial adaptor. In some aspects, the multi-chamber device includes a barrel defining a first chamber and a second chamber fluidly isolated from each other; and a plunger assembly having a first plunger rod with a distal end extendable with respect to the first chamber, and a second plunger rod with a distal end extendable with respect to the chamber. In some aspects, a portion of the first plunger rod is movable independently of a portion of the second plunger rod to advance the distal end of first plunger rod with respect to the barrel a first extent and to advance the distal end of the second plunger rod with respect to the barrel a second extent different from the first extent. In some aspects, the vial is adaptor configured to be releasably coupled with the multi-chamber device to fluidly couple a separate chamber with the first chamber of the barrel.

In some aspects, the second plunger rod includes a proximal plunger rod and a distal plunger rod axially movable with respect to each other; and the proximal plunger rod and the first plunger rod are coupled together to move together.

In some aspects, the multi-chamber system further includes an injection system engageable with the multi-chamber device. In some aspects, the multi-chamber device includes one or more engaging elements; the injection system includes one or more engaging elements configured to engage with the one or more engaging elements of the multi-chamber device; and the one or more engaging elements of the multi-chamber device are radially movable between an engaged position engaged with the one or more engaging elements of the injection system, and a disengaged position in which the multi-chamber device is axially movable with respect to the injection system. In some aspects, the multi-chamber system further includes an adaptor system. In some aspects, the adaptor system includes one or more engaging element configured to engage with the one or more engaging elements of the injection system, and radially movable between an engaged position engaged with the one or more engaging elements of the injection system, and a disengaged position in which the multi-chamber device is axially movable with respect to the injection system.

In accordance with various principles of the present disclosure, a method of separately ejecting material from separate chambers within a multi-chamber device includes advancing a first plunger rod of the multi-chamber device with respect to a first chamber of the multi-chamber device a first extent; and advancing a second plunger rod of the multi-chamber device with respect to a second chamber of the multi-chamber device a second extent different from the first extent. In some aspects, a portion of the second plunger rod is coupled with the first plunger rod to advance with the first plunger rod.

In some aspects, advancing the second plunger rod includes advancing a proximal portion of the second plunger rod with respect to a distal portion of the second plunger rod.

In some aspects, the method further includes retracting the first plunger rod and the second plunger rod proximally with respect to the multi-chamber device barrel to engage the proximal portion of the second plunger rod with the distal portion of the second plunger rod to be advanceable together.

In some aspects, advancing the first plunger rod includes advancing a distal end of the first plunger rod with respect to the barrel a first distance; and advancing the second plunger rod includes advancing a distal end of the second plunger rod with respect to the barrel a second distance less than the first distance.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary.

For example, devices may be enlarged so that detail is discernable, but is intended to be scaled down in relation to facilitate injection into a patient. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of a system for combining and/or delivering injectable materials to a patient formed in accordance with various principles of the present disclosure.

FIG. 1A is a detail of FIG. 1.

FIG. 2 illustrates an exploded perspective view of an example of an embodiment of multi-chamber system such as illustrated in FIG. 1.

FIG. 3A-FIG. 3I illustrate various stages of preparing an injectable material, in accordance with various principles of the present disclosure, with an example of an embodiment of a multi-chamber system such as illustrated in FIG. 2.

FIG. 3Ex illustrates a cross-sectional view along line IIIEx-IIIEx of FIG. 3E.

FIG. 3Fx illustrates a cross-sectional view along line IIIFx-IIIFx of FIG. 3F.

FIG. 3Fs illustrates a detail view of FIG. 3F.

FIG. 3Hx illustrates a cross-sectional view along line IIIHx-IIIHx of FIG. 3H.

FIG. 4A illustrates a perspective view of a multi-chamber device such as illustrated in

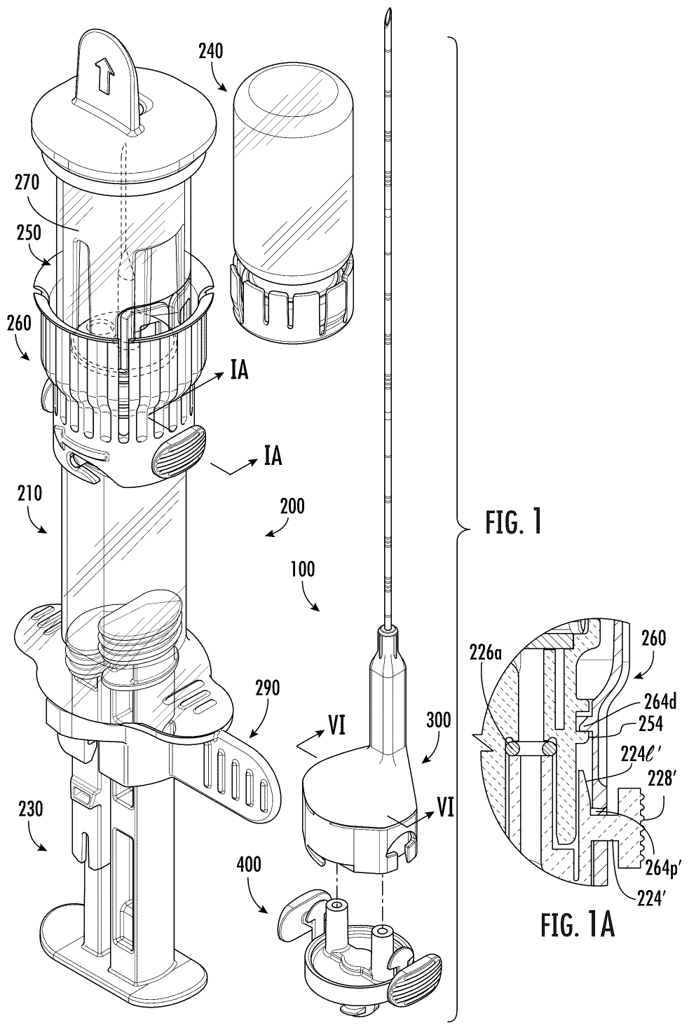

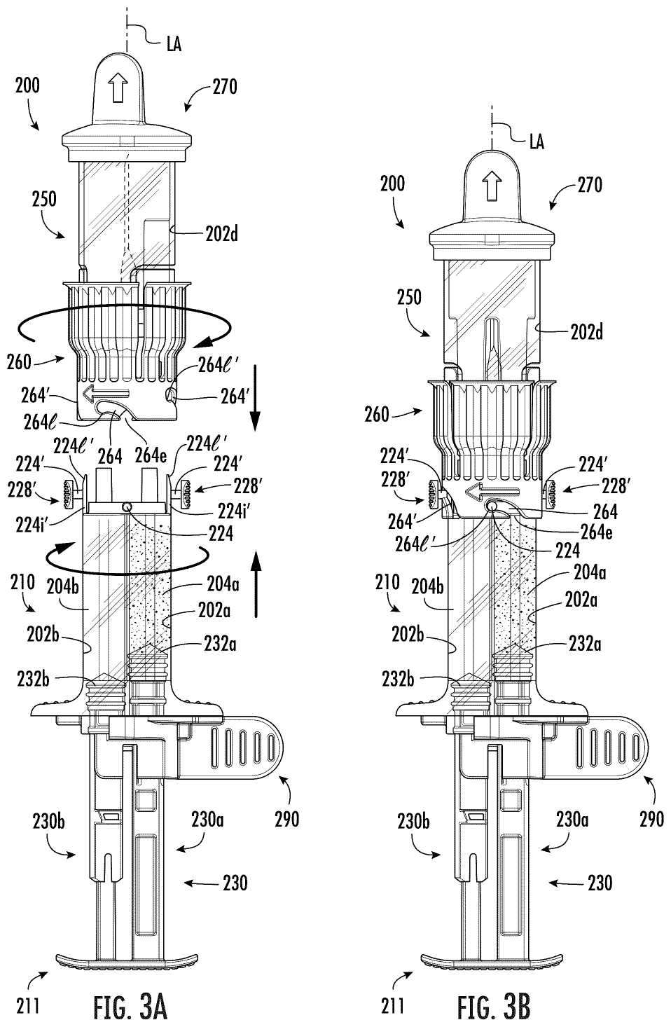

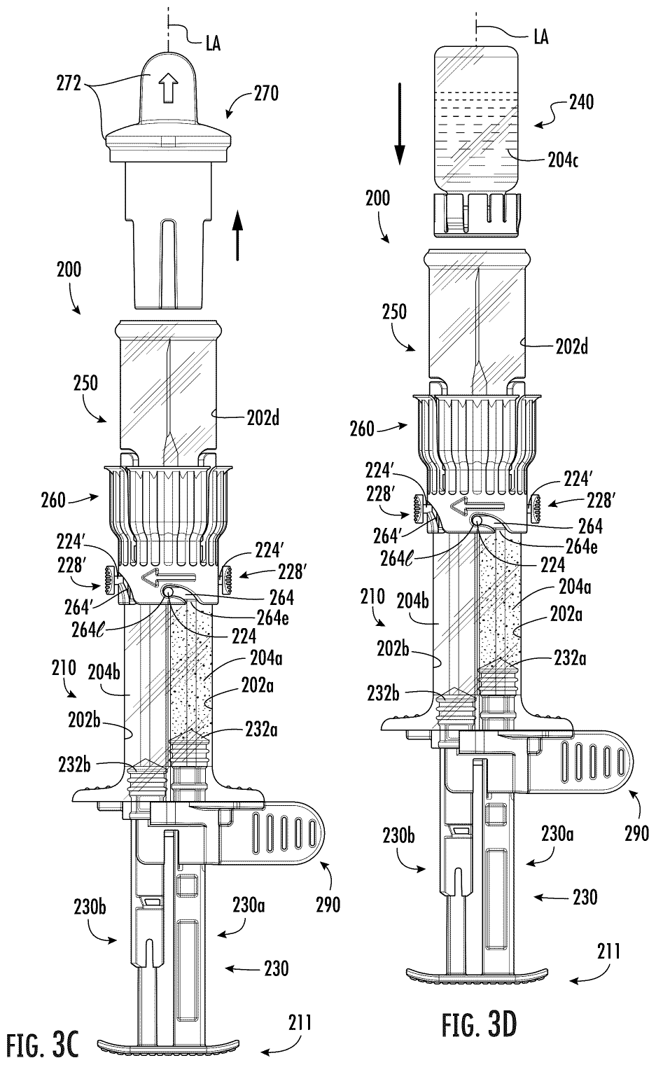

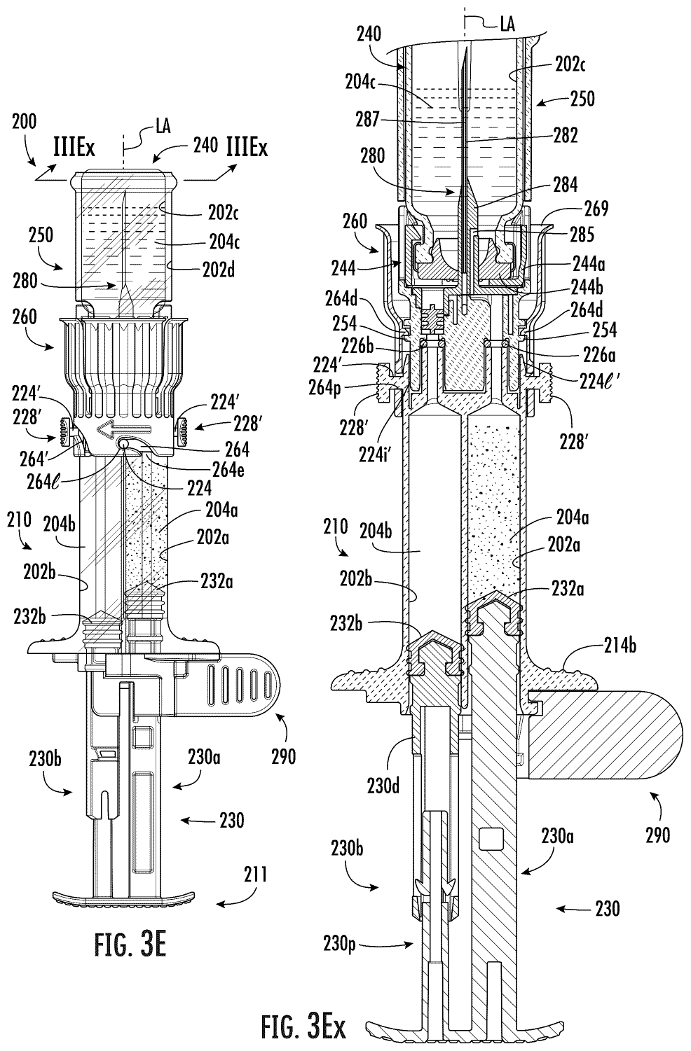

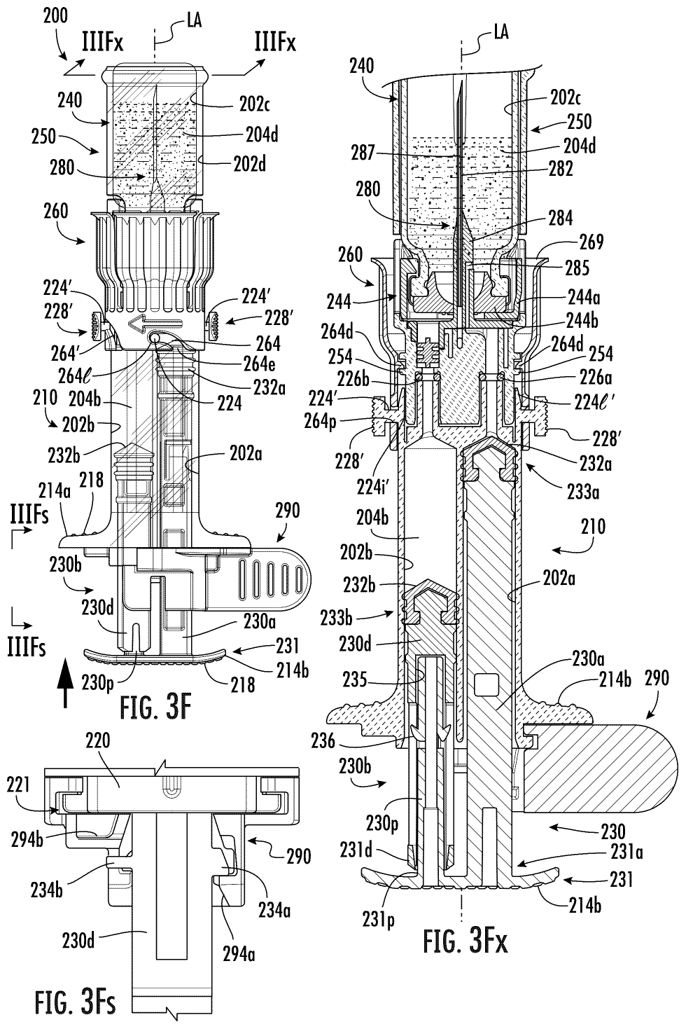

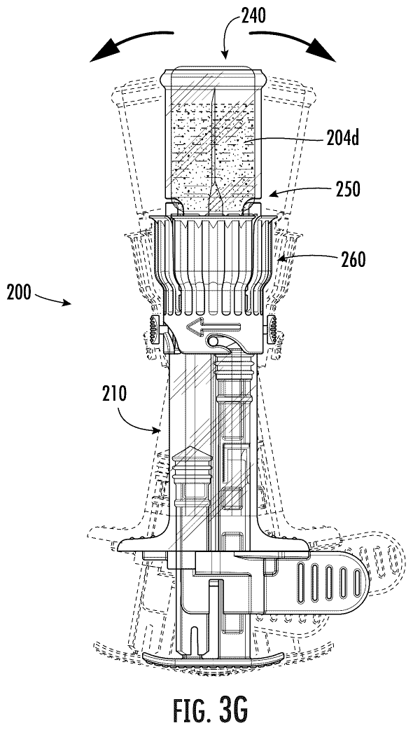

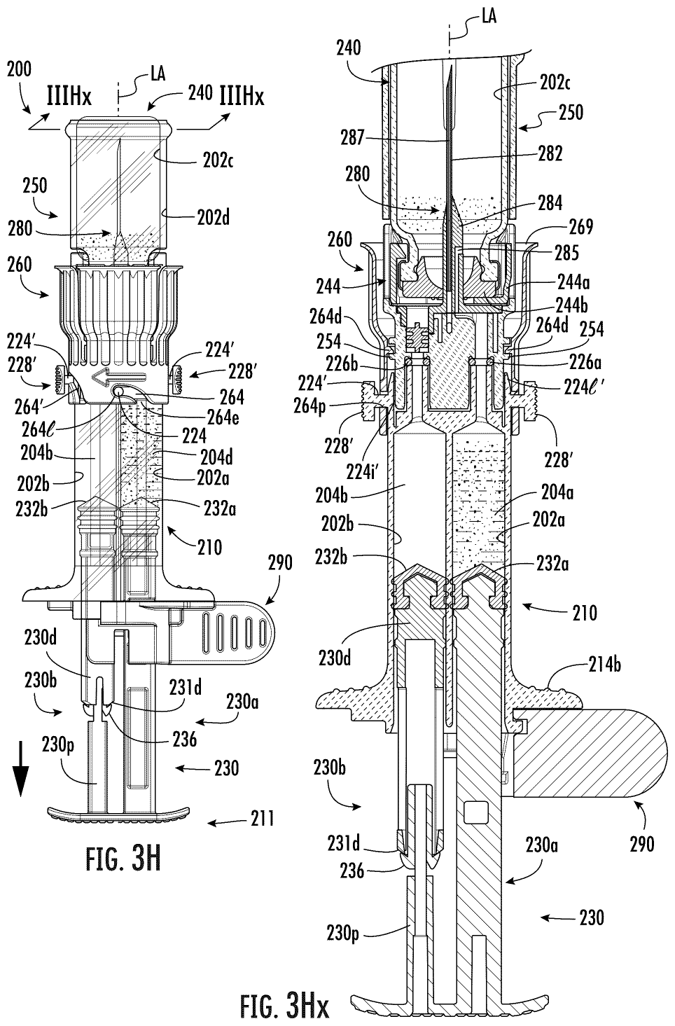

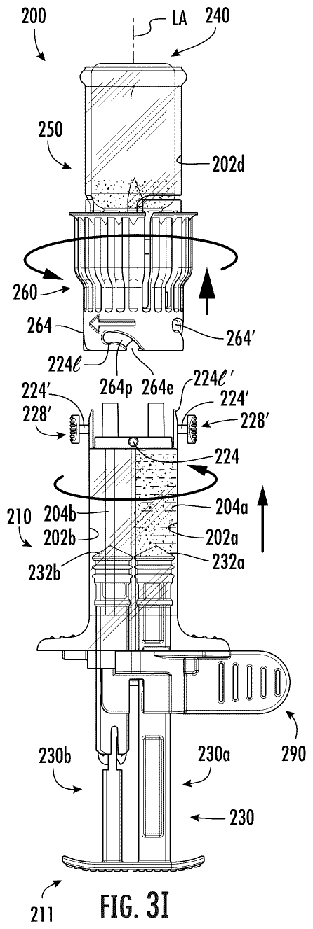

FIG. 3H positioned for engagement with an injection system such as illustrated in FIG. 1.

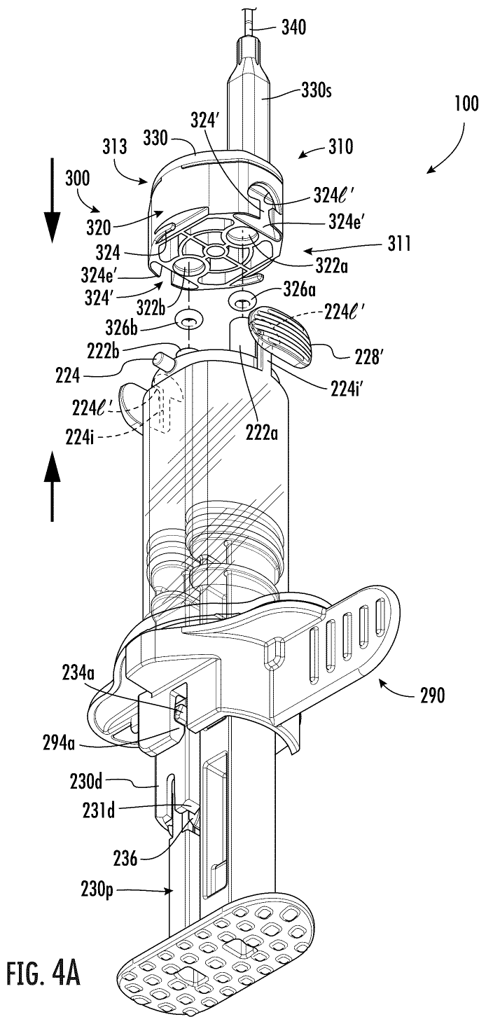

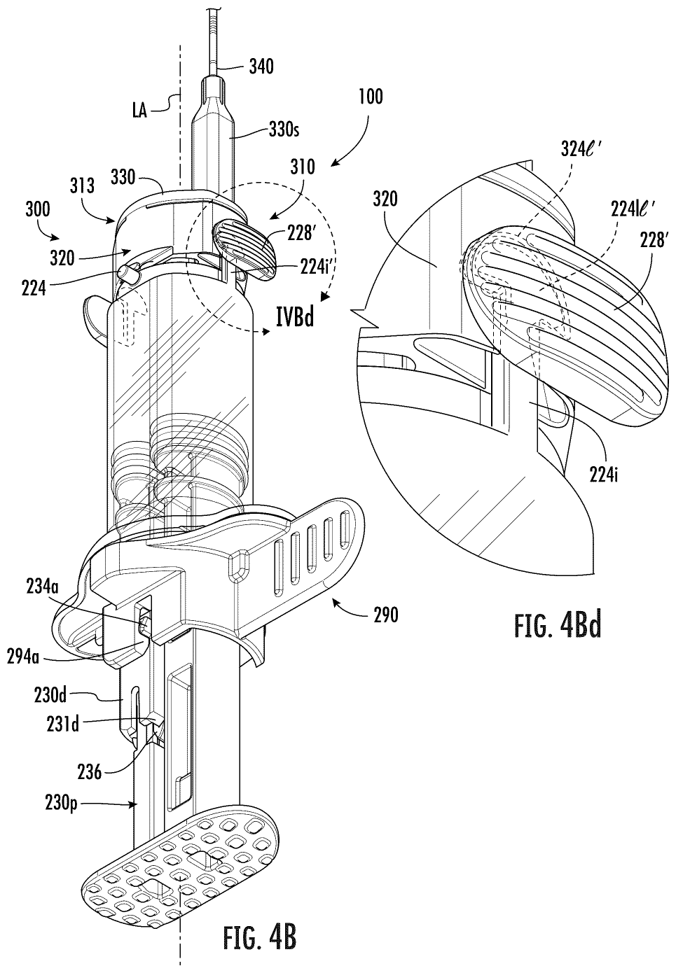

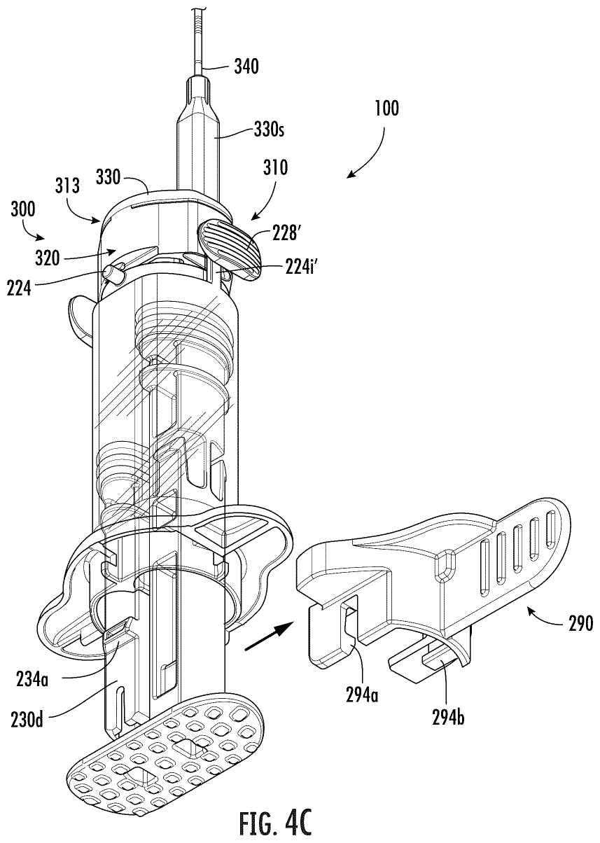

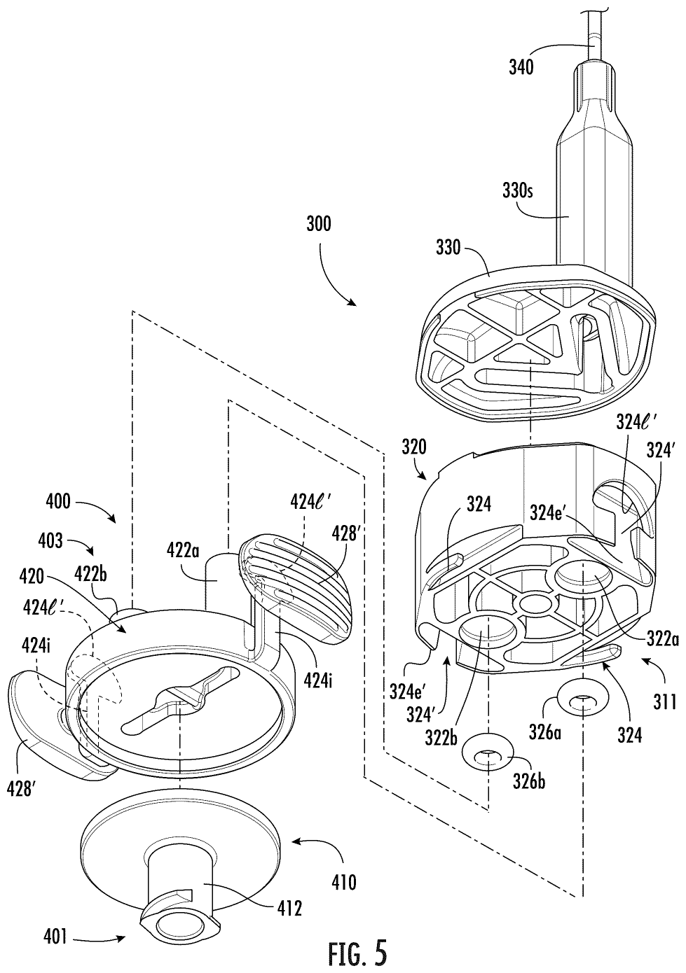

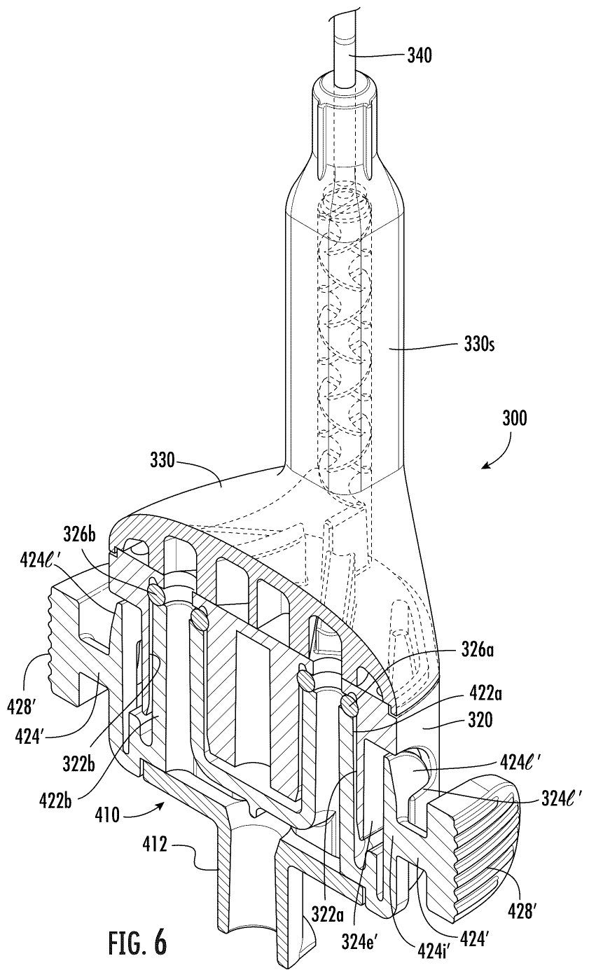

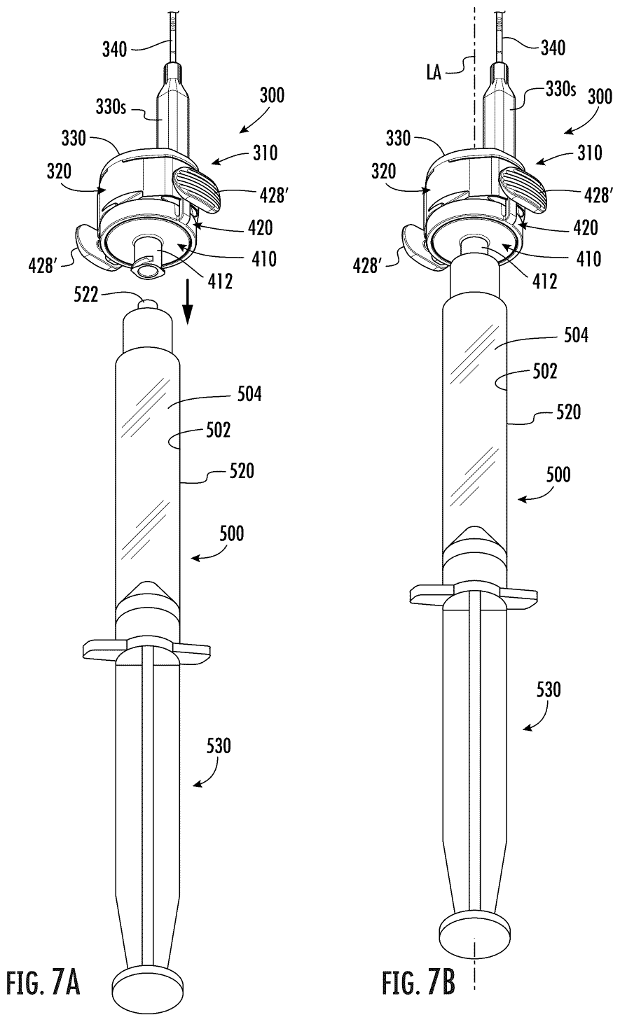

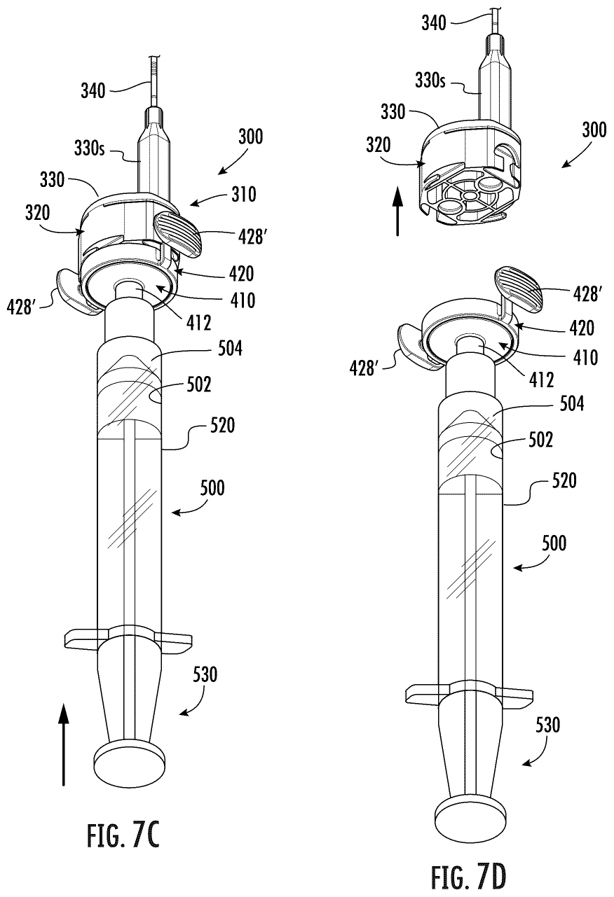

FIG. 4B illustrates the multi-chamber device and injection system illustrated in FIG. 4A fluidly coupled for delivery of injectable material, from within the multi-chamber device and via the injection system, to a patient.

FIG. 4Bd is a detail of FIG. 4B.

FIG. 4C illustrates the plunger assembly of the multi-chamber device illustrated in

FIG. 4B advanced to deliver injectable material out from the multi-chamber device and through the injection system for delivery to a patient.

FIG. 5 illustrates an injection system such as illustrated in FIG. 4A-FIG. 4C in position to be coupled with an adaptor system such as illustrated in FIG. 1.

FIG. 6 illustrates a cross-sectional view along line VI-VI of FIG. 1.

FIG. 7A-FIG. 7D illustrate various stages of connecting a syringe with an adaptor system and an injection system such as illustrated in FIG. 1 and FIG. 5, injecting the preliminary injectable material (e.g., into a patient), and detaching the syringe after delivery of the injectable material to the patient.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an clement is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location.

Various medical procedures involve delivery (e.g., injection) of injectable material(s) into the body before, during, or after the procedure. Preferably, the injectable material is biocompatible, and optionally biodegradable. The injectable material may serve a variety of purposes, including, without limitation, differentiating tissue (e.g., by creating a “bleb” or other raised or swelled region to distinguish an anatomical region), spacing anatomical structures from one another, otherwise affecting (e.g., shielding, coating, covering, modifying, etc.) an anatomical structure, etc. It will be appreciated that the term “tissue” is a broad term that encompasses a portion of or site within a body: for example, a group of cells, a group of cells and interstitial matter, an organ, a portion of an organ, an anatomical portion of a body, e.g., a rectum, ovary, prostate, nerve, cartilage, bone, brain, or portion thereof, etc. Moreover, reference may be made, herein, to a “target” in referring to an area of the patient's body at which a procedure is to be performed. However, it will be appreciated that such reference is to be broadly understood and is not intended to be limited to tissue or to particular procedures. Finally, reference may be made to target tissue, target location, target site, target tissue site, anatomical site, delivery site, deployment site, injection site, treatment site, etc., including combinations thereof and other grammatical forms thereof, interchangeably and without intent to limit.

Certain specific aspects of the present disclosure relate to preparing an injectable material for placement between target tissue to be treated and other tissues. For the sake of convenience, and without intent to limit, reference is made to an injectable material, such as a filler, including, without limitation, a gel composition. The injectable material may be delivered within the patient to displace the tissue relative to a tissue that is to be treated by a therapeutic procedure or otherwise (e.g., not necessarily therapeutic). The injectable material may displace and/or shield a tissue to protect the tissue against possible side effects of treatment of a target tissue, such as the effects of a treatment involving radiation or cryotherapy. In some aspects, the injectable material may displace anatomical tissue and/or may increase the distance between the target tissue and other tissues. For instance, if the target tissue is to be irradiated, the injectable material may space other tissues from the target tissue so that the other tissues are exposed to less radiation and/or are shielded from the radiation. In some aspects, the injectable material is injectable as a filler in a space between tissues. A first tissue may then be treated by radiation, while the injectable material reduces passage of radiation therethrough into a second tissue. The first tissue may be irradiated while the second tissue, spaced by the injectable material, receives less radiation than it would have in the absence of the injectable material. An effective amount of an injectable material may be injected into a space between a first tissue to be treated and a second tissue which can be a critically sensitive organ. For instance, in the context of treatment of prostate cancer, an injectable material may be injected into the Denonvilliers' space (a region between the rectum and prostate) to create additional space between the rectum and prostate and/or to shield the rectum during treatment, thereby reducing rectal radiation dose and associated side effects.

In some aspects of the present disclosure, components of an injectable material are combined by a system formed in accordance with various principles of the present disclosure, for injection into or near a target site. It will be appreciated that terms such as combine, mix, blend, etc., (including other grammatical forms thereof) may be used interchangeably herein without intent to limit unless otherwise indicated. Reference is accordingly made herein to a combining system generically, without intent to specifically require active combining/mixing.

In accordance with various principles of the present disclosure, the injectable material may be a filler such as a hydrophilic polymer, a gel, a hydrogel, etc. For instance, the injectable material can include polymeric materials which are capable of forming a hydrogel upon crosslinking. Optionally, the polymer forms a hydrogel within the body. A hydrogel is defined as a substance formed when a polymer (natural or synthetic) is crosslinked via covalent, ionic, or hydrogen bonds to create a three-dimensional structure which entraps water molecules to form a gel. Naturally-occurring and synthetic hydrogel-forming polymers, polymer mixtures, and copolymers may be utilized as hydrogel precursors. In some aspects, the hydrogel can be formed by a composition formed by two or more constituents/components (e.g., mixing accelerant fluid, diluent, and polyethylene glycol (PEG) together), and may include one or more polysaccharide compounds or a salt thereof. For example, the composition may include a cellulose compound such as carboxymethyl cellulose (CMC) or salt thereof (e.g., CMC sodium), xanthan gum, alginate or a salt thereof (e.g., calcium alginate, such as Ca-alginate beads), chitosan, and/or hyaluronic acid. In some examples, the composition may comprise a mixture of hyaluronic acid and CMC, and/or may be crosslinked with a suitable crosslinking compound, such as butanediol diglycidyl ether (BDDE). In some aspects, the polysaccharide may be a homopolysaccharide or a heteropolysaccharide.

In some aspects of the present disclosure, two or more components of an injectable material are provided separately, and are combined by a device, system, and method in accordance with various principles of the present disclosure to form a compound material to be injected into or near a target site by devices, systems, and methods in accordance with various principles of the present disclosure. The injectable material may be delivered within the patient to displace the tissue relative to a tissue that is to be treated by a therapeutic procedure or otherwise (e.g., not necessarily therapeutic). A composition to be injected into a patient may be a combination of two or more components combined by a device, system, or method formed in accordance with various principles of the present disclosure. For instance, devices, systems, or methods of the present disclosure may be used to combine a first component and a second component for injection into a patient. The first component may be a precursor, e.g., a first component to be combined with a second component to form the injectable compound. The second component may be an accelerator, an accelerant, an activating agent, a cross-linking inducing agent, a catalyst, an initiator, etc., which, upon combination with the precursor, produces the injectable compound, such as by altering the chemical composition or structure of the first component. The components may be combined prior to (e.g., immediately prior to or during) delivery (e.g., e.g., during injection) to the patient so that the injectable material does not have time to form into a structure which may be difficult to inject or otherwise deliver/administer to the patient. As such, the combination of the first component and the second component may be such that the injectable compound attains its final desired properties/reaches its final form in situ.

In some embodiments, the injectable material is formed of a first component, a second component, and a third component. For instance, for various reasons it may be desirable to provide a first, precursor component in a solid form (e.g., to allow mixing at the time of delivery, and/or to be more stable for storage and/or transport). The first component is combinable with the third component, and the thus-formed combined composition (which may be referenced as the precursor) is then combinable with the second component once the medical professional is ready to deliver (e.g., inject) the injectable material to the patient. The second component may facilitate a crosslinking interaction between the first and third components, for example, by initiating or accelerating the crosslinking interaction of the first and third components. Typically, one or more of the components of the injectable materials are biocompatible polymers. In some aspects, one of the first component or third component is a reactive polymer, such as a cross-linkable and/or hydrophilic polymer component (e.g., PEG), and the other of the first component or third component is a diluent (e.g., mostly water) in which a solid or semi-solid form of the one of the first component or third component is dissolved or dispersed, and/or with which the one of the first component or third is cross-linked (or at least cross-linkable, such as upon further combination with the second component), to form a precursor. The second component may be an accelerator, an accelerant, an activating agent, a catalyst, an initiator, etc. (such terms being used interchangeably herein without intent to limit), combinable and reactive with the precursor (formed from the first component and the third component) to form the desired injectable material. In one example of an embodiment, a first component, in the form of a cross-linking agent (specifically, trilysine, which contains multiple nucleophilic groups, specifically, amino groups), is mixed with a third component, in the form of a reactive polymer (specifically, PEG) that has been derivatized with reactive, optionally electrophilic, groups (specifically, succinimide ester groups), under acidic pH conditions where the succinimide ester groups and the amino groups do not react to any significant degree. When this mixture is combined with a second component, in the form of an accelerant (specifically, a basic buffer solution), the pH of the resulting mixture becomes basic, at which point the amino groups of the trilysine react with the succinimide ester groups of the PEG to form covalent bonds, thereby crosslinking the PEG and forming a hydrogel.

It will be appreciated that reference to “first”, “second”, or “third” is not intended to connote a particular nature of the material or the order in which the material is combined. As such, “first”, “second”, and “third” may be used to reference any of three components forming an injectable material in accordance with various principles of the present disclosure. A non-limiting example of such components combinable by devices, systems, or methods in accordance with various principles of the present disclosure includes a first component such as a reactive component, a solute, etc.; a second component such as a diluent with which the reactive component is to be combined to form a precursor; and a third component such as an accelerator combinable with the precursor to form an injectable material. The injectable material is a biocompatible material, such as a polymeric material, such as a filler, or such as a hydrogel.

In some examples, the composition may be or include a gel with a desired gel strength and/or viscosity, such as a biocompatible gel suitable for injection (e.g., through a needle), as discussed in further detail below. In one example of an embodiment, the one of the components is a biocompatible polymeric component. More particularly, in one example of an embodiment, one of the components is a hydrophilic polymer, which may be natural or synthetic in origin, and may be anionic, cationic, zwitterionic, or neutrally charged. Non-limiting examples of hydrophilic polymers include natural hydrophilic polymers including proteins such as collagen and polysaccharides such as gellan gum, xanthan gum, gum arabic, guar gum, locust bean gum, alginate, and carrageenans, and synthetic hydrophilic polymers such as polyethylene glycols (PEG), PEG-methacrylates, PEG-methylmethacrylates, polyvinyl alcohols, polyacrylates and polymethacrylates, polyacrylic acids and their salts, polymethacrylic acids and their salts, polymethylmethacrylates, carboxymethylcelluloses, hydroxyethylcelluloses, polyvinylpyrrolidones, polyacrylamides such as N,N-methylene-bis-acrylamides or tris (hydroxymethyl) methacrylamides. The hydrophilic polymer may be modified to provide functional groups that are reactive with functional groups of a suitable cross-linking agent, which may be a covalent or ionic cross-linking agent.

The concentrations of gelling agent(s) in a composition formed in accordance with various principles of the present disclosure maybe at least about 0.01% by weight with respect to the total weight of the composition, and at most about 2.0% by weight with respect to the total weight of the composition, including increments of about 0.01% therebetween. For instance, the concentration of gelling agent(s) may range from about 0.02% to about 1.5%, from about 0.05% to about 1.0%, from about 0.05% to about 0.50%, from 0.05% to about 0.15%, from about 0.10% to about 0.20%, from about 0.15% to about 0.25%, from about 0.20% to about 0.30%, from about 0.25% to about 0.35%, from about 0.30% to about 0.40%, from about 0.35% to about 0.45%, from about 0.40% to about 0.50%, from about 0.1% to about 0.5%, or from about 0.1% to about 0.15% by weight with respect to the total weight of the composition. In at least one example, the total concentration of the gelling agent(s) in the composition may range from about 0.05% to about 0.5% by weight with respect to the total weight of the composition.

In some examples, a composition formed in accordance with various principles of the present disclosure may have a viscosity of at least about 0.001 pascal-second (Pa·s), and at most about 0.100 Pa·s at a shear rate of 130 s⁻¹. For instance, the composition may have a viscosity ranging from about 0.005 Pa·s to about 0.050 Pa·s, from about 0.010 Pa·s to about 0.050 Pa·s, from about 0.010 Pa·s to about 0.030 Pa·s, from about 0.010 Pa·s to about 0.020 Pa·s, from about 0.020 Pa·s to about 0.030 Pa·s, or from about 0.020 Pa·s to about 0.040 Pa·s at a shear rate of 130 s⁻¹. Thus, for example, the composition may be or comprise a gel having a viscosity of about 0.005

Pa·s, about 0.006 Pa·s, 0.008 Pa·s, about 0.010 Pa·s, about 0.011 Pa·s, about 0.012 Pa·s, about 0.013 Pa·s, about 0.014 Pa·s, about 0.015 Pa·s, about 0.016 Pa·s, about 0.017 Pa·s, about 0.018 Pa·s, about 0.019 Pa·s, about 0.020 Pa·s, about 0.022 Pa·s, about 0.024 Pa·s, about 0.026 Pa·s, about 0.028 Pa·s, about 0.030 Pa·s, about 0.032 Pa·s, about 0.034 Pa·s, about 0.036 Pa·s, about 0.038 Pa·s, about 0.040 Pa·s, about 0.042 Pa·s, about 0.044 Pa·s, about 0.046 Pa·s, about 0.048 Pa·s, or about 0.050 Pa·s at a shear rate of 130 s⁻¹. In at least one example, the composition may have a viscosity greater than 0.0050 Pa·s at a shear rate of 130 s⁻¹, e.g., a viscosity ranging from about 0.005 Pa·s to about 0.050 Pa·s, at a shear rate of 130 s⁻¹. In at least one example, the composition may have a viscosity greater than 0.010 Pa·s at a shear rate of 130 s⁻¹, e.g., a viscosity ranging from about 0.010 Pa·s to about 0.030 Pa·s, at a shear rate of 130 s⁻¹.

Alternatively or additionally, a composition formed in accordance with various principles of the present disclosure may have a viscosity of at least about 0.001 Pa·s, and at most about 0.050 Pa·s at a shear rate of 768 s⁻¹. For instance, the composition may have a viscosity ranging from about 0.002 Pa·s to about 0.030 Pa·s, from about 0.003 Pa·s to about 0.020 Pa·s, from about 0.004 Pa·s to about 0.010 Pa·s, from about 0.004 Pa·s to about 0.006 Pa·s, from about 0.005 Pa·s to about 0.007 Pa·s, from about 0.006 Pa·s to about 0.008 Pa·s, from about 0.007 Pa·s to about 0.009 Pa·s, or from about 0.008 Pa·s to about 0.01 Pa·s at a shear rate of 768 s⁻¹. Thus, for example, the composition may be or comprise a gel having a viscosity of about 0.003 Pa·s, about 0.004 Pa·s, about 0.005 Pa·s, about 0.006 Pa·s, about 0.007 Pa·s, about 0.008 Pa·s, about 0.009 Pa·s, or about 0.010 Pa·s at a shear rate of 768 s⁻¹. In at least one example, the composition may have a viscosity less than 0.010 Pa·s at a shear rate of 768 s⁻¹, e.g., a viscosity ranging from about 0.005 Pa·s to about 0.009 Pa·s at a shear rate of 768 s⁻¹. In at least one example, the composition may have a viscosity ranging from about 0.004 Pa·s to about 0.010 Pa·s at a shear rate of 768 s⁻¹. Further, for example, the composition may have a viscosity ranging from about 0.010 Pa·s to about 0.030 Pa·s, e.g., about 0.017 Pa·s at a shear rate of 130 s⁻¹ and a viscosity ranging from about 0.004 Pa·s to about 0.010 Pa·s, e.g., about 0.007 Pa·s, at a shear rate of 768 s⁻¹.

The various components forming the injectable material may be kept separate until the time of the procedure in which the final composition is used for various reasons, such as to maintain stability of the individual components. In some embodiments, a multi-chamber system includes separate chambers for components to be combined to form the injectable material to be delivered to the patient by an injection system. In some embodiments, a first component and a second component are separately contained within a first chamber and a second chamber, respectively, of a multi-chamber device. A third component may be contained in a separate device defining a third chamber of the multi-chamber system. To deliver the injectable material, the components of the first and third chambers are combined within the first chamber (e.g., to form a precursor), and then the components of the first and second chambers and injected together into the patient. The multi-chamber device may or may not mix the contents of the first chamber with the contents of the second chamber. The final form, structure, composition, properties, etc., of the injectable material may be attained once the combined components are within the patient.

The present disclosure provides devices, systems, and methods for combining components to form an injectable material, or at least a precursor for combination to form an injectable material. and corresponding medical devices, systems, and methods for use thereof and/or delivery to a treatment site of a patient. According to some aspects of the present disclosure, such as described above, a multi-chamber system may include a plurality of chambers for the one or more components of the injectable material and for combinations of such components. It will be appreciated that terms such as chamber, reservoir, container, vial, lumen, etc., may be used interchangeably herein without intent to limit, to refer to elements which contain, convey, hold, transport, collect, etc., a component (fluid, particulate, liquid, solid, gas, etc.) of an injectable material. Suitable chambers may include, for example, vials, syringes (e.g., a syringe barrel compatible with a manual or automatic injection system) and other fluid containers, such as configured for use with a suitable injection system. Examples of materials suitable for chambers of devices or systems of the present disclosure include, but are not limited to, cyclic olefin polymer, polypropylene, polycarbonate, polyvinyl chloride, and glass. In some aspects, one of these materials (e.g., cyclic olefin copolymer specifically) can have a coating applied to it (such as SiO₂ coating), which is advantageous so the coating can provide a primary oxygen barrier, behave as a glass-like layer, and/or can be applied using a vapor deposition process.

A combining device or system formed in accordance with various principles of the present disclosure to combine two or more components to form an injectable material may be removably connected to one or more injection systems which are configured to deliver the injectable materials to a patient. According to some aspects of the present disclosure, the filler compositions which may be used with various systems disclosed herein, e.g., the compositions prepared by the various devices, systems, methods disclosed herein, may have sufficient strength, e.g., gel strength, to withstand the forces on the continuity of the three-dimensional configuration (e.g., gel network) of the composition, and thereby minimize the effects of such forces. In the meantime, compositions with sufficient strength to withstand forces thereon may have a viscosity suitable for injection, e.g., a viscosity that does not cause the composition to become stuck in the reservoir(s), delivery lumen, needle, or other structure in which the composition is contained or through which it passes.

According to some aspects of the present disclosure, the composition may maintain its three-dimensional structure until the composition is injected into a patient (e.g., through a needle), whereupon the structure may form fragments of the original continuous, three-dimensional network. Those fragments may have a diameter corresponding to the diameter of the lumen through which it passes into the patient (e.g., the lumen of an injection needle), such that the fragments are as large as possible in-vivo to retain as much of the three-dimensional structure of the composition as possible. Injection of these larger-sized particles or fragments is believed to increase the amount of time the gel remains within the tissue.

In some examples, the injection system includes a needle. In some embodiments, the needle may be a hypodermic needle, and may range from a size of 7-gauge (4.57 mm outer diameter (OD), 3.81 mm inner diameter (ID)) to 33-gauge (0.18 mm OD, 0.08 mm ID), e.g., a size of 16-gauge (1.65 mm OD, 1.19 mm ID), 18-gauge, 21-gauge (0.82 mm OD, 0.51 mm ID), 22-gauge (0.72 mm OD, 0.41 mm ID), 23-gauge (0.64 mm OD, 0.33 mm ID), or 24-gauge (0.57 mm OD, 0.31 mm ID). According to some aspects of the present disclosure, the size of the needle may be chosen based on the viscosity and/or components of the composition, or vice versa. According to some aspects of the present disclosure, the size of the needle may be 23-gauge or 25-gauge. In some cases, a larger size of 18-gauge, 20-gauge, 21-gauge, or 22-gauge may be used to inject the compositions disclosed herein. Examples of materials which may be used to form the needle include, but are not limited to, metals and metal alloys, such as stainless steel and Nitinol, and polymers. The distal tip of the needle may be sharpened, and may have a beveled shape. The proximal end of the needle may include a suitable fitting/adaptor (e.g., a Luer adapter) for engagement with a syringe or other reservoir. In some examples, the needle may include an elongated tube or catheter between the needle tip and the proximal fitting/adapter.

As noted above, compositions used with systems disclosed herein may have large particulate matter (relative to the injection system lumen) and/or a high viscosity for passage through a lumen sized to inject the material into the patient. The amount of force required to move the composition through a needle aperture (generally described as “peak load” force) may depend on the viscosity of the composition, the dimensions of the needle (inner diameter, outer diameter, and/or length), and/or the material(s) from which the needle is formed. For example, a greater amount of force may be applied to inject the composition through a 33-gauge needle in comparison to a 7-gauge needle. Additional factors that may affect the amount of force applied to inject the composition may include the dimensions of a catheter (inner diameter, outer diameter, and/or length) connecting the mixing system to the needle. Suitable peak loads for injection with one or two hands may range from about 5 lb·f to about 25 lb·f, such as from about 10 lb·f to about 20 lb·f, e.g., about 15 lb·f. The loads measured for a given gel concentration may vary for different needles and flow rates.

According to some aspects of the present disclosure, a combining device or system can be included in a kit for introducing an injectable material into a patient, whereby the injectable material can include any of a variety of suitable compositions. Kits or systems may be configured to store one or more of the components of a composition until the medical professional is ready to mix the composition for delivery to a patient. For instance, compositions, such as hydrogels, may be prepared so that the precursor(s) and any related activating agent(s) are stored in the kit with diluents as may be needed. Applicators may be used in combination with the same. Kits formed in accordance with various principles of the present disclosure can be manufactured using medically acceptable conditions and contain components that have sterility, purity, and preparation that is pharmaceutically acceptable. Solvents/solutions may be provided in the kit or separately.

The kit may include one or more syringes and/or needles for mixing and/or delivery of the injectable material, and/or for additional aspects of the procedure in which the injectable material is to be used. The kit or system may comprise various components as set forth herein. For instance, a target site into which an injectable material is to be delivered may be pre-treated using one or more components of the kit. One example of a pretreatment includes hydrodissection, such as with saline, to create space for injectable material to be injected at or in the vicinity of the target tissue site. Once saline has been injected to the treatment site, a combining device or system can be connected to a needle (e.g., an 18-gauge spinal needle) to then deliver the injectable material to the treatment site. For instance, in treating prostate cancer, a 5-10 mm layer of filler (e.g., gel composition) may be injected along the posterior wall of the prostate between the prostate and rectum. Once the filler has been injected into the space between the rectum and prostate, ultrasound images can be obtained.

In accordance with various principles of the present disclosure, a combining and/or delivery system is configured to facilitate combining of components of an injectable material. In some aspects, the injectable material is a combination of a first component, a second component, and a third component, such as described above. In accordance with various principles of the present disclosure, a combining and/or delivery system includes various components, devices, systems, etc., and associated methods configured to facilitate combining of material in a first injectable material transfer device with a second injectable material transfer device. The first injectable material transfer device defines a chamber containing a component of an injectable material, and the second injectable material transfer device a separate chamber containing a separate component of the injectable material. The first injectable material transfer device and the second injectable material transfer device are fluidly coupled for combining of the components thereof. The components may be combined within one or both of the chambers of the first and second injectable material transfer devices. In some embodiments, the components are combined to form a precursor, and the combining and/or delivery system has yet another chamber containing an accelerant, an accelerator, an activating agent, a cross-linking inducing agent, a catalyst, an initiator, etc. (such terms may be used interchangeably herein, without intent to limit, reference generally being made to an accelerant for the sake of convenience and without intent to limit). In some embodiments, the first injectable material transfer device has a barrel defining a chamber therein, and a plunger assembly configured to advance towards/into the barrel chamber to eject a material from the barrel chamber and/or to retract away from the barrel chamber to aspirate materials into the barrel chamber. In some embodiments, the other injectable material transfer device is a vial, and the adaptor is a vial adaptor configured to facilitate fluid coupling of the vial with the first injectable material transfer device.

In accordance with various principles of the present disclosure, a combining and/or delivery system is configured to facilitate combining of components of an injectable material. In some aspects, the injectable material is a combination of a first component, a second component, and a third component, such as described above. A combining and/or delivery system formed in accordance with various principles of the present disclosure facilitates combining/mixing of the various components of an injectable material. Additionally or alternatively, a combining and/or delivery system formed in accordance with various principles of the present disclosure facilitates delivery of the injectable material to an injection system configured to deliver to and/or deposit the injectable material into (e.g., inject) a patient (e.g., to a target site within the patient). More particularly, various aspects of the present disclosure simplify assembly, alignment, mixing, dispensing, etc., of an injectable material which is combined or blended or mixed from separate components before delivery to a patient.

A multi-compartment combining and/or delivery system of the present disclosure may include an injectable material transfer device having a barrel defining at least one chamber therein, and a plunger assembly moveable (e.g., advanceable and/or retractable) with respect to the at least one chamber to transfer (e.g., eject, aspirate, purge, etc.) material with respect to the at least one chamber. In some aspects, the plunger assembly has multiple positions defining multiple actuation stages. In some aspects, the barrel of the injectable material transfer device defines more than one chamber therein. In some aspects, a multi-chamber injectable material transfer device has a plunger assembly with more than one plunger rod. In some aspects, each plunger rod is movable with respect to a corresponding chamber defined within the barrel chamber. In some aspects, the plunger assembly includes a rod for each chamber of a multi-chamber injectable material transfer device. In some aspects, a multi-rod plunger assembly has multiple positions defining multiple actuation stages.

In accordance with various principles of the present disclosure, a multi-chamber injectable material transfer device has a barrel defining a first chamber and a second chamber, and a plunger assembly defining a first rod movable with respect to the first barrel chamber and a second rod movable with respect to the second barrel chamber. The distal end of each of the first and second rods may be movable independently of the other distal end. In some aspects, the rods of a multi-rod multi-stage plunger assembly of a multi-chamber injectable material transfer device are configured to be advanced or retracted with respect to their respective chambers independently of one another. For instance, at least one rod of a multi-rod plunger assembly of a multi-chamber injectable material transfer device may be configured to eject and/or aspirate a first component with respect to a first chamber (in which the first component is contained) to a different extent than another rod of the multi-rod plunger assembly ejects/aspirates material with respect to another chamber. In some instances, a first rod of a multi-rod plunger assembly of a multi-chamber injectable material transfer device ejects a first amount of material from a first chamber, and a second rod of the multi-rod plunger assembly ejects a second amount of material from a o-rsecond chamber, the second amount being different from the first amount. In some instances, the second rod ejects a limited amount, such as a determined amount, of a second component from a second chamber with respect to which the second rod moves, while the first rod ejects more (e.g., most) of the first component within the first chamber with respect to which the first rod moves. In some instances, the first rod without ejects and/or aspirates material with respect to the first chamber, whereas the second rod only ejects, or only aspirates, or does not eject or aspirate material with respect to a second chamber. In some aspects, the individual rods of a multi-stage plunger assembly have multiple positions defining multiple actuation stages independent of one another. In some aspects, the individual rods of a multi-stage plunger assembly are movable independently from one another as well as together with one another. In some embodiments, a retainer is positioned on the plunger assembly to limit movement of the plunger assembly with respect to the barrel of the injectable material transfer device, such as to prevent inadvertent ejection of materials therefrom.

Various embodiments of combining and/or delivery devices, systems, and methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features in the drawings are identified by common reference characters and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). Finally, certain features in one embodiment may be used across different embodiments and are not necessarily individually labeled when appearing in different embodiments.

Turning now to the drawings, an example of an embodiment of a combining and/or delivery system 100 formed in accordance with various principles of the present disclosure is illustrated in FIG. 1. The combining and/or delivery system 100 includes a multi-chamber system 200 and an injection system 300. The multi-chamber system 200 may be used to transport components of an injectable material to a facility/location at which the injectable material is to be delivered into a patient. For the sake of convenience, and without intent to limit, reference may be made to delivery by injection into the patient (e.g., the injectable material is delivered to the patient by being injected into the patient), although the present disclosure need not be so limited. The material combined and/or delivered by a combining and/or delivery system 100 of the present disclosure may be any other type of material to be administered to a patient, such as other than by injection. The injectable material may, as described above, include two or more components which are to remain separate until the time of the procedure. Once a medical professional is ready to inject the injectable material into the patient, the components of the injectable material are combined by the combining and/or delivery system 100 to be combined into (e.g., to react with each other to form) a compound material having the desired characteristics for being delivered to and/or deposited at a target site by the combining and/or delivery system 100. The multi-chamber system 200 may be fluidly coupled with the injection system 300 of the combining and/or delivery system 100 to combine the injectable material and/or to deliver the injectable material to the patient. The injection system 300 may be usable independently of the multi-chamber system 200. An optional adaptor system 400 may be included with the combining and/or delivery system 100 to adapt the injection system 300 for use with an off-the-shelf (e.g., pre-existing, separately sold, commercially available, etc.) device used to perform a procedure, treatment, pretreatment, etc., prior to or after delivery of the injectable material to the target site, as described in further detail below.

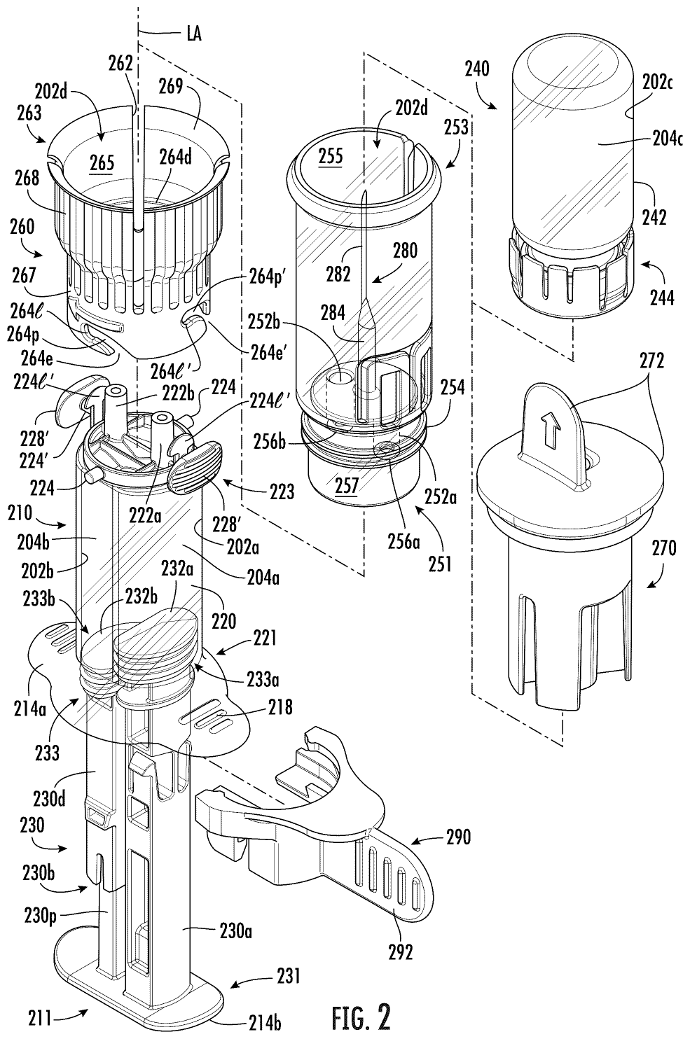

Various elements of the multi-chamber system 200 may be appreciated with reference to FIG. 2. The multi-chamber system 200 includes a device with at least one chamber for containing a component of an injectable material to be delivered to a patient. In the example of an embodiment illustrated in FIG. 2, the multi-chamber system 200 includes a multi-chamber device 210 having a barrel 220 defining a first chamber 202a and a second chamber 202b of the multi-chamber system 200. In some embodiments, the chambers 202a, 202b have substantially symmetrical cross-sectional shapes, such as half-circles if within a barrel 220 having a substantially circular cross-section, although the configurations need not be so limited. For instance, the chambers 202a, 202b may have different volumes, cross-sectional shapes, etc., such as may be dictated by the properties of the components to be contained therein. The first chamber 202a is defined to be separate and fluidly-isolated from the second chamber 202b. For instance, the contents of the first chamber 202a, e.g., a first component 204a of an injectable material, may react with the contents of the second chamber 202b, e.g., a second component 204b of an injectable material, when combined. In some embodiments, the first component 204a is combined with a third component 204c of the injectable material, such as to form a precursor 204d, and the second component 204b may react with the precursor 204d (e.g., to accelerate reaction of the first component 204a and the third component 204c). It may be desirable to keep these components separate until ready for delivery to a patient. To avoid unintentional premature combination, the contents of the chambers 202a, 202b are maintained separate from each other by the fluid isolation of the chambers 202a, 202b. When (e.g., only when) a medical professional is ready to deliver the injectable material (e.g., as a combined injectable material), the components within the chambers 202a, 202b may be combined and allowed to react with each other. The various devices, systems, and method of the present disclosure prevent unintentional combination of the first component 204a and the second component 204b, such as before the medical professional is ready to deliver the injectable material (to be formed from the first component 204a and the second component 204b) to a patient as a combined injectable material. Additionally or alternatively, the various devices, systems, and method of the present disclosure facilitate case and accuracy of combining of components to form an injectable material at the appropriate time.

The example of an embodiment of a multi-chamber device 210 illustrated in FIG. 2 further includes a plunger assembly 230 extending proximally from the proximal end 221 of the barrel 220 (towards the proximal end 211 of the multi-chamber device 210). The plunger assembly 230 is movable, such as axially/longitudinally slidable (e.g., along the longitudinal axis LA), with respect to the barrel 220 to move (e.g., eject) materials out of and/or to move (e.g., aspirate) materials into chambers within the barrel 220. In the illustrated example of an embodiment, the plunger assembly 230 has a first plunger rod 230a movable/slidable with respect to the first chamber 202a and a second plunger rod 230b movable/slidable with respect to the second chamber 202b. In the illustrated example of an embodiment, distal movement of the plunger rods 230a, 230b moves materials out of the first chamber 202a and the second chamber 202b, respectively, via a first nozzle 222a and a second nozzle 222b, respectively, extending distally from the distal end 223 of the barrel 220. Conversely, proximal movement of the plunger rods 230a, 230b moves materials into the first chamber 202a and the second chamber 202b, respectively, via the first nozzle 222a and the second nozzle 222b, respectively. It will be appreciated that the plunger rods 230a, 230b may move independently of each other, such described in further detail below. Optionally, the first plunger rod 230a has a plunger stopper 232a at the distal end 233a thereof, and/or the second plunger rod 230b has a plunger stopper 232b at the distal end 233b thereof. The plunger stoppers 232a, 232b may be formed of an appropriate scaling material (e.g., an elastomeric material) known to those of ordinary skill in the art for retaining the first component 204a within the first chamber 202a and the second component 204b within the second chamber 202b.

The multi-chamber system 200 optionally includes a third chamber 202c for containing an additional, third component 204c. The third component 204c may be combinable with the first component 204a contained within the first chamber 202a. The third chamber 202c optionally is defined in a separate device 240, formed and provided separately from the multi-chamber device 210, such as illustrated in FIG. 2. For the sake of convenience, and without intent to limit, the separate device is referenced herein as a vial 240, having a vial body 242 defining the third chamber 202c (which may alternately be referenced herein as a vial chamber 202c), and a vial cap 244 closing an open end 241 of the vial body 242 to contain (e.g., seal) the third component 204c within the third chamber 202c. The vial cap 244 may be a two-part cap, with an outer cap 244a holding a plug or stopper 244b with respect to the open end 241 of the vial body 242, as may be appreciated with reference to FIG. 3Ex. The stopper 244b may be formed of a suitable material (e.g., an elastic material) known to those of ordinary skill in the art as capable of scaling the third component 204c within the third chamber 202c defined within the vial 240 and optionally also allowing access therethrough, as described in further detail below.

A vial adaptor 250 is configured to facilitate holding/coupling of the vial 240 with respect to the multi-chamber device 210 to transfer materials therebetween, in a manner described in further detail below. The example of an embodiment of a vial adaptor 250 illustrated in FIG. 2 defines a fourth chamber 202d of the multi-chamber system 200 opening to the distal end 253 of the vial adaptor 250. The fourth chamber 202d may be configured as a vial-receiving chamber configured to receive the vial 240 and to hold the vial 240 in alignment with the multi-chamber device 210 so that the multi-chamber device 210 and the vial 240 may be fluidly coupled for transferring and/or exchanging material therebetween. For instance, the vial adaptor 250 may facilitate alignment and/or fluid sealing between the vial adaptor 250 and the multi-chamber device 210 so that materials may be transferred between the third chamber 202c within a vial 240 (within the vial adaptor 250) and the multi-chamber device 210 without leakage, and/or without significant manipulation of components of the combining and/or delivery system 100. More particularly, the vial adaptor 250 facilitates fluid coupling of a first chamber 202a of a multi-chamber device 210 formed in accordance with various principles of the present disclosure with a separate chamber 202c, and the combination of the components 204a, 204c contained within the chambers 202a, 202c. The vial adaptor 250 may include a first nozzle port 252a positioned and configured to be fluidly coupled with (e.g., to receive and to be in fluid communication with) the first nozzle 222a of the barrel 220 of the multi-chamber device 210, and a second nozzle port 252b positioned and configured to be fluidly coupled with (e.g., to receive and to be in fluid communication with) the second nozzle 222b of the barrel 220 of the multi-chamber device 210, as may be appreciated with reference to FIG. 3Ex and as discussed in further detail below.

The example of an embodiment of a multi-chamber system 200 illustrated in FIG. 2 further includes a sleeve lock 260 configured to facilitate secure coupling of the vial adaptor 250 with the multi-chamber device 210. For instance, the example of an embodiment of a sleeve lock 260 illustrated in FIG. 2 has a plurality of engaging elements 264, 264′ configured to facilitate engagement/coupling and/or disengagement/decoupling of the sleeve lock 260 with respect to the multi-chamber device 210 and the vial adaptor 250. The sleeve lock 260 may be configured to stabilize the multi-chamber device 210 and the vial adaptor 250 with respect to each other, such as to maintain fluid coupling therebetween without leakage. For instance, the sleeve lock 260 may be generally cylindrical, such as in the shape of a ring or collar, and may alternately be referenced as a locking ring, locking collar, adaptor ring, adaptor collar, etc., without intent to limit. The length of the sleeve lock 260 (e.g., along the longitudinal axis LA) may be selected to hold the vial adaptor 250 against lateral movement (transverse to the longitudinal axis LA) with respect to the multi-chamber device 210 when coupled thereto.

Optionally, the example of an embodiment of a sleeve lock 260 illustrated in FIG. 2 is configured to be mounted with respect to (e.g., and carried on) the vial adaptor 250. For instance, the example of an embodiment of a sleeve lock 260 illustrated in FIG. 2 has a distal end 263 configured to be carried on the vial adaptor 250, for engagement/coupling with and/or disengagement/decoupling of the sleeve lock 260 and vial adaptor 250 (as coupled together) with respect to a multi-chamber device 210. In the example of an embodiment of a sleeve lock 260 as illustrated FIG. 1 and FIG. 2, the sleeve lock 260 has one or more distal engaging elements 264d in the form of one or more circumferentially-extending features (e.g., undercut locking tab features, ribs, shoulders, etc.) extending partially or fully around the interior surface 265 of the sleeve lock 260, and the vial adaptor 250 has one or more corresponding sleeve-lock-engaging elements 254 in the form of corresponding features (e.g., a groove, rib, circumferentially-extending shoulder, etc.) extending partially or fully circumferentially around the vial adaptor 250. The distal engaging elements 264d on the sleeve lock 260 are configured to engage with the sleeve-lock-engaging elements 254 on the vial adaptor 250 in a manner which rotatably couples the sleeve lock 260 and the vial adaptor 250. More particularly, as may be appreciated with reference to FIG. 2, and the detail FIG. 1A of FIG. 1, the illustrated example of an embodiment includes distal engaging elements 264d on the sleeve lock 260 in the form of one or more (e.g., 1-4, or more) circumferential ribs extending radially inwardly from the interior surface 265 of the sleeve lock 260 and configured to be engageable with engaging elements 254 on the vial adaptor 250 in the form of one or more corresponding circumferential grooves 254 defined in the interior surface 255 of the vial adaptor 250. The illustrated examples of embodiments of engaging elements 264d, 254 may extend partially or fully around the sleeve lock 260 and vial adaptor 250, respectively. Such rotational coupling between the sleeve lock 260 and the vial adaptor 250 facilitate engagement of the proximal engaging elements 264p, 264p′ of the sleeve lock 260 with respect to the sleeve-lock-engaging elements 224, 224′ of the multi-chamber device 210, as described in further detail below.

Optionally, the sleeve lock 260 includes flex slots 262 facilitating mounting of the sleeve lock 260 on the vial adaptor 250, such as by an interference fit or snap fit. The flex slots 262 may also facilitate optional removal of the sleeve lock 260 from the vial adaptor 250, such as by radially expanding the distal end 263 of the sleeve lock 260 to radially expand the distal engaging elements 264d to be disengaged from (e.g., by being moved axially with respect to) the sleeve-lock-engaging elements 254 on the vial adaptor 250. In some embodiments, the distal end 263 of the sleeve lock 260 include radially-outwardly extending gripping sections 269 configured to facilitate flexing of portions of the sleeve lock 260 with respect to the flex slots 262.

It will be appreciated that the distal engaging elements 264d of the illustrated example of an embodiment of a sleeve lock 260 may not be at the distal-most end of the sleeve lock 260, and may simply be distal to proximal engaging elements 264p, 264p′ configured to be engageable with corresponding sleeve-lock-engaging elements 224, 224′ on the barrel 220 of the multi-chamber device 210, as described in further detail below. The sleeve lock 260 may extend distally beyond the distal engaging clement 264d to define a fifth chamber 202e of the multi-chamber system 200 configured to receive at least a portion of the vial adaptor 250 therein.

In accordance with various principles of the present disclosure, as may be appreciated with reference to FIG. 2, and as noted above, the engaging elements 264 of the illustrated example of an embodiment of a sleeve lock 260 also include engaging elements 264p, 264p′ configured to facilitate engagement of the sleeve lock 260 (already coupled with the vial adaptor 250) with the multi-chamber device 210. More particularly, in the illustrated example of an embodiment of a multi-chamber system 200, proximal engaging elements 264p, 264p′ of the sleeve lock 260 are configured to facilitate ready engagement/coupling and/or disengagement/decoupling of the proximal end 261 of the sleeve lock 260 with respect to sleeve-lock-engaging elements 224, 224′ associated with the distal end 213 of the multi-chamber device 210. For instance, in the illustrated example of an embodiment of a multi-chamber system 200 illustrated in FIG. 1 and FIG. 2, the proximal engaging elements 264p, 264p′ on the sleeve lock 260 and the sleeve-lock-engaging elements 224, 224′ at the distal end 213 of the multi-chamber device 210 (e.g., associated with the distal end 223 of the barrel 220) are configured to be readily engaged and/or disengaged by axial and/or rotational movements, in contrast with interference fits which generally require overcoming of a threshold force (such as between the sleeve lock 260 and the vial adaptor 250). The sleeve-lock-engaging elements 224, 224′, 264p, 264p′ on the multi-chamber device 210 may be configured to readily be initially engaged with respect to, and/or to remain engaged with, and/or to be disengaged from the proximal engaging elements 264p, 264p′ on the sleeve lock 260.

In the example of an embodiment of a multi-chamber system 200 illustrated in FIG. 2, the proximal engaging elements 264p, 264p′ of the sleeve lock 260 may be in the form of slots, and the sleeve-lock-engaging elements 224 of the multi-chamber device 210 may be in the form of radially-outwardly extending bosses, projections, pins, nubs, etc. (referenced herein as projections for the sake of convenience and without intent to limit) configured to engage with the proximal engaging elements 264p, 264p′. Such configuration allows a relatively simple (e.g., a bayonet-type) connection of the sleeve lock 260 (with the vial adaptor 250 coupled thereto) with respect to the multi-chamber device 210 facilitating ready engagement and/or disengagement of the vial adaptor 250 (and thus a vial 240 inserted or to be inserted therein) with respect to the multi-chamber device 210). It will be appreciated that the proximal engaging elements 264p, 264p′ of the sleeve lock 260 may not all be at the same axial position along the sleeve lock 260 in order to engage with sleeve-lock-engaging elements 224, 224′ of the multi-chamber device 210 which are not all at the same axial position with respect to the multi-chamber device 210/barrel 220, for reasons described in further detail below.

It will be appreciated that the proximal engaging elements 264p, 264p′ of the sleeve

lock 260 may be configured to facilitate fluid coupling of the multi-chamber device 210 with the vial adaptor 250 in addition to maintaining coupling of the multi-chamber device 210 and the vial adaptor 250 once brought into fluid communication. For instance, the sleeve lock 260 (with the vial adaptor 250 optionally already mounted on the sleeve lock 260, such as described above) may be brought together generally axially to fluidly couple the barrel nozzles 222a, 222b with the vial adaptor nozzle ports 252a, 252b as well as to engage the sleeve-lock-engaging elements 224, 224′ of the multi-chamber device 210 with the proximal engaging elements 264p, 264p′ of the sleeve lock 260, such as by entering the entry openings 264e, 264e′ of the slot-like proximal engaging elements 264p, 264p′ illustrated in FIG. 2. The sleeve lock 260 and the multi-chamber device 210 may be rotated with respect to each other to cause the sleeve-lock-engaging elements 224 of the multi-chamber device 210 to extend further into the proximal engaging elements 264p, 264p′ of the sleeve lock 260 to interlock the sleeve-lock-engaging elements 224, 224′ and the proximal engaging elements 264p, 264p′ to hold the vial adaptor 250 axially in place with respect to the multi-chamber device 210 via the sleeve lock 260. As may be appreciated, the rotational mounting of the sleeve lock 260 with respect to the vial adaptor 250 allows the barrel nozzles 222a, 222b and the vial adaptor nozzle ports 252a, 252b, to remain in fluid communication, as the sleeve lock 260 is rotated into a locking position (inhibiting axial separation of the multi-chamber device 210 and the vial adaptor 250) without disturbing the seating of the nozzles 222a, 222b and nozzle ports 252a, 252b with respect to one another. In some embodiments surface features 268 (e.g., axial grooves or ribs, dimples, bumps or other projections, etc.) may be provided on the exterior surface 267 of the sleeve lock 260 to facilitate grasping and rotating of the sleeve lock 260 with respect to the multi-chamber device 210 (and optionally with respect to the vial adaptor 250 as well).

The vial adaptor 250 may be coupled with the multi-chamber device 210 with a protective cap 270 positioned within the fourth chamber 202d (defined within the vial adaptor 250), prior to insertion of the vial 240 into the fourth chamber 202d. The protective cap 270 may be configured to protect or shield components of a fluid exchange device 280 extending within the fourth chamber 202d of the vial adaptor 250 (such as a needle 282 and fluid exchange spike 284, as illustrated in FIG. 2). The protective cap 270 may also protect the vial adaptor 250 from entry of unwanted matter or debris from entering the fourth chamber 202d.

The protective cap 270 is removed from the fourth chamber 202d to allow insertion of the vial 240 into the fourth chamber 202d. Once the vial 240 is seated within the fourth chamber 202d, the fluid exchange device 280 fluidly communicates the first chamber 202a within the barrel 220 of the multi-chamber device 210 with the third chamber 202c within the vial 240, as described in further detail below. As described above, once the multi-chamber device 210 and the vial 240 are fluidly communicated, the components 202a, 202c, respectively therein, may be combined, exchanged, etc., such as to form a precursor 204d.

The various interactions of the various components of a multi-chamber system 200 formed in accordance with various principles of the present disclosure and illustrated in FIG. 2 may be appreciated with reference to FIG. 3A-FIG. 3I. The multi-chamber device 210 and the vial adaptor 250 are illustrated in FIG. 3A in position to be coupled together, such as with the assistance of the sleeve lock 260. The optional protective cap 270 is illustrated in FIG. 3A positioned within the fourth chamber 202d of the vial adaptor 250. Additionally, a retainer 290 is illustrated as coupled with respect to the proximal end 221 barrel 220 (e.g., mounted thereon), such as by a sliding engagement of engaging ribs, shoulders, etc., (e.g., in a direction transverse to the longitudinal axis LA), such in a manner such as known those of ordinary skill in the art, the present disclosure not being limited in this regard. The retainer 290 may be configured and positioned to limit various movements of the plunger assembly 230 with respect to the barrel 220, such as to prevent inadvertent ejection of materials from the multi-chamber device 210, as described in further detail below.

The nozzles 222a, 222b of the multi-chamber device 210 are aligned with the nozzle ports 252a, 252b of the vial adaptor 250 to be fluidly communicated with one another. If the sleeve-lock-engaging elements 224, 224′ of the multi-chamber device 210 and the proximal engaging elements 264p, 264p′ of the sleeve lock 260 are not aligned for engagement with one another (e.g., the sleeve-lock-engaging elements 224, 224′ are not positioned to be moved axially into engagement with the entry openings 264e, 264e′ of the slot-like proximal engaging elements 264p, 264p′), then the rotatable mounting of the sleeve lock 260 on the vial adaptor 250 allows the sleeve lock 260 to be rotated (e.g., with respect to the vial adaptor 250) into position for engagement of the engaging elements 224, 224′, 264p, 264p′ with respect to one another. The vial adaptor 250 and the multi-chamber device 210 may then be brought together, along the axial directional arrow illustrated in FIG. 3A, from a position as illustrated in FIG. 3A, to be fluidly coupled together such as illustrated in FIG. 3B.

Axial movement of the multi-chamber device 210 and the sleeve lock 260 together also allows the sleeve-lock-engaging elements 224, 224′ of the multi-chamber device 210 to be moved axially into the entry openings 264e, 264e′ of the slot-like proximal engaging elements 264p, 264p′. The sleeve lock 260 and the multi-chamber device 210 are then rotated with respect to each other along the circumferentially-extending directional arrows illustrated in FIG. 3A to move the sleeve-lock-engaging elements 224, 224′ of the multi-chamber device 210 further into the proximal engaging elements 264p, 264p′ of the sleeve lock 260. The illustrated examples of embodiments of proximal engaging elements 264p, 264p′ extend generally axially and circumferentially from the entry openings 264e, 264e′ thereof to generally-axially-extending locking sections 264 [symbol] , 264 [symbol] ′ at the end of the circumferential extents of the proximal engaging elements 264p, 264p′. As the sleeve-lock-engaging elements 224, 224′ are moved towards the locking sections 264 [symbol] , 264 [symbol] ′, the multi-chamber device 210 and the sleeve lock 260 (with the vial adaptor 250 coupled thereto) are axially drawn closer together. Relative rotation of the sleeve lock 260 and the multi-chamber device 210 is continued until the sleeve-lock-engaging elements 224, 224′ of the multi-chamber device 210 are engaged with locking sections 264 [symbol] , 264 [symbol] ′ of the proximal engaging elements 264p, 264p′ of the sleeve lock 260. A biasing element may be provided to bias the multi-chamber device 210 and the sleeve lock 260 apart from each other to retain the sleeve-lock-engaging elements 224, 224′ in engagement with the locking sections 264 [symbol] , 264 [symbol] ′ to inhibit unintended relative rotation of the multi-chamber device 210 and the sleeve lock 260 (which may lead to decoupling of the multi-chamber device 210 and the sleeve lock 260). For instance, seals 226a, 226b (e.g., O-rings) may fluidly seal the fluid coupling of the nozzles 222a, 222b and nozzle ports 252a, 252b (such as shown in FIG. 3EX, described in further detail below) may also bias the multi-chamber device 210 apart from the vial adaptor 250 and the sleeve lock 260 which is coupled thereto.

To prepare the multi-chamber system 200 for combining the first component 204a therein with a separately delivered third component 204c, the protective cap 270 is separated from the vial adaptor 250 (e.g., removed from the fourth chamber 202d), as illustrated in FIG. 3C. Optionally, the protective cap 270 includes one or more grasping sections 272, such as a radially-outwardly projecting flange or an axially-extending flange, configured to facilitate grasping and pulling on the protective cap 270 to remove the protective cap 270 from the vial-receiving fourth chamber 202d defined in the vial adaptor 250. The vial 240 is then aligned with the vial adaptor 250 for insertion into the fourth chamber 202d, as illustrated in FIG. 3D.

In FIG. 3E, the vial 240 is illustrated positioned within the fourth chamber 202d of the vial adaptor 250. The vial adaptor 250 may be configured to facilitate alignment of the vial 240 with respect to the multi-chamber device 210 to align the vial 240 with the fluid exchange device 280 between the vial 240 and the multi-chamber device 210. The fluid exchange device 280 may include a piercing element 282, such as a needle, extending from a fluid exchange spike 284 configured to fluidly communicate the first chamber 202a and the third chamber 202c. As may be appreciated with reference to FIG. 3Ex, showing a cross-sectional view along line IIIEx-IIIEx in FIG. 3E, the cap 244 of the vial 240 may have an outer cap 244a extending around the circumference/periphery of a stopper 244b leaving a generally central region of the stopper 244b exposed for access therethrough, such as by the piercing element 282 of the fluid exchange device 280. The first chamber 202a and the third chamber 202c of the multi-chamber system 200 may be fluidly communicated via a fluid exchange lumen 285 extending through the fluid exchange spike 284, such as described in further detail in co-pending provisional patent application, ______, titled DEVICES, SYSTEMS, AND METHODS FOR COMBINING AND/OR DELIVERING INJECTABLE MATERIALS, and filed on even date herewith [ATTORNEY DOCKET 2001.3125100], which application also is hereby incorporated by reference herein in its entirety and for all purposes.

With the vial 240 seated with respect to the vial adaptor 250, and the third chamber 202c (defined within the vial 240) in fluid communication with the first chamber 202a (defined within the multi-chamber device 210), such as illustrated in FIG. 3E and FIG. 3Ex, the multi-chamber device 210 may be actuated to advance the plunger assembly 230 distally with respect to the barrel 220, as illustrated in FIG. 3F and FIG. 3Fx. In some embodiments, the multi-chamber device 210 includes grips 214 facilitating actuation of the multi-chamber device 210. For instance, the example of an embodiment of a multi-chamber device 210 illustrated in FIG. 2, FIG. 3F, and FIG. 3Fx includes a finger grip 214a (e.g., one or more radially-outwardly extending flanges) on the barrel 220 and a finger grip 214b (e.g., one or more radially-outwardly extending flanges) on the plunger assembly 230 to facilitate grasping and/or holding of the multi-chamber device 210 to facilitate moving the plunger assembly 230 relative to the barrel 220. In some embodiments, the finger grip 214a and/or the finger grip 214b is provided with surface features 218 to facilitate manual engagement/gripping thereof by the finger or fingers or hand or hands of a medical professional moving the barrel 220 and plunger assembly 230 with respect to each other.

As noted above with reference to FIG. 2, the multi-chamber device 210 has a barrel 220 with first and second chambers 202a, 202b defined therein, and a plunger assembly 230 with first and second plunger rods 230a, 230b movable/slidable with respect to the first and second chambers 202a, 202b. In accordance with various principles of the present disclosure, as may be appreciated with reference to FIG. 3F and the cross-sectional view along line IIIFx-IIIFx thereof, as illustrated in FIG. 3Fx, the plunger assembly 230 may be configured such that at least a portion of the first plunger rod 230a moves independently of at least a portion of the second plunger rod 230b. As such, the first plunger rod 230a and the second plunger rod 230b do not necessarily advance together with respect to their respective barrel chambers 202a, 202b, and can eject different amounts of material from their respective barrel chambers 202a, 202b. For instance, in

FIG. 3F and FIG. 3Fx, the illustrated example of an embodiment of a plunger assembly 230 allows the first plunger rod 230a to eject the first component 204a out of the first chamber 202a and into the vial 240, while the second plunger rod 230b ejects some (less than the ejected amount of the first component 204a) or none of the second component 204b out of the second chamber 202b. For instance, in the example of an embodiment illustrated in FIG. 3Fx, the distal end 233a of the first plunger rod 230a (e.g., the plunger stopper 232a, if provided) is extended the full, or close to the full, longitudinal extent of the first chamber 202a (along a longitudinal axis LA, such as of, or substantially parallel to, a longitudinal axis of the barrel 220) to expel all or most of the first component 204a out from the first chamber 202a and to inject the first component 204a into the third chamber 202c for combination with the third component 204c therein. In contrast, the distal end 233b of (e.g., the plunger stopper 232a, if provided, on) the second plunger rod 230b in the example of an embodiment illustrated in FIG. 3Fx is only partially advanced distally into the second chamber 202b when the first plunger rod 230a is substantially fully advanced. In other words, the second plunger rod 230b may be advanced along only a portion of the longitudinal extent of the second chamber 202b (along the longitudinal axis LA, typically shorter than the longitudinal extent along which the first plunger rod 230a advances. Partial advancement of the second plunger rod 230b may be beneficial and/or desirable to expel any surplus of the second component 204b, and/or any air bubbles, out from the second chamber 202b into a purge reservoir within the vial adaptor 250 (such as described in further detail in co-pending provisional patent application ______, titled DEVICES, SYSTEMS, AND METHODS FOR COMBINING AND/OR DELIVERING INJECTABLE MATERIALS, and filed on even date herewith [ATTORNEY DOCKET 2001.3128100], which application is hereby incorporated by reference herein in its entirety and for all purposes). The purge reservoir may collect the purged material within the multi-chamber device 210 so that such material is not expelled outside the multi-chamber device 210 (e.g., onto the medical professional, the patient, the floor, etc.).

In accordance with various principles of the present disclosure, the second plunger rod 230b has a proximal plunger rod 230p movable with respect to a distal plunger rod 230d. Thus, although the proximal plunger rod 230p is coupled (e.g., fixed) with respect to the first plunger rod 230a to move therewith, such as via the finger grip 214b of the plunger assembly 230 (as illustrated in FIG. 3F), the distal plunger rod 230d is movable with respect to the proximal plunger rod 230p and thus also with respect to the first plunger rod 230a. As such, distal advancement of the first plunger rod 230a (e.g., from the position illustrated in FIG. 3E to the position illustrated in FIG. 3F) distally advances the proximal plunger rod 230p therewith. However, as may be appreciated with reference to FIG. 3Fx, the proximal plunger rod 230p may, at least with initial distal movement of the first plunger rod 230a, advances distally with respect to the distal plunger rod 230d without also distally advancing the distal plunger rod 230d. In the illustrated example of an embodiment of a multi-part second plunger rod 230b, the proximal plunger rod 230p is telescopically mounted within a receiving channel 235 defined generally axially through the distal plunger rod 230d. As the first plunger rod 230a moves the proximal plunger rod 230p distally from the position illustrated in FIG. 3E and FIG. 3Ex to the position illustrated in FIG. 3F and FIG. 3Fx, the proximal plunger rod 230p moves distally within the distal plunger rod 230d without causing the distal plunger rod 230d to advance distally therewith. As such, the first plunger rod 230a injects the first component 204a into the third chamber 202c, while the second plunger rod 230b does not eject the second component 204b from the second chamber 202b. Optionally, the relative lengths of the proximal plunger rod 230p, the distal plunger rod 230d, and the first plunger rod 230a are such that the proximal plunger rod 230p advances the distal plunger rod 230d distally over only a portion of the distal travel of the first plunger rod 230a, such as may be appreciated upon comparison of the positions of the distal ends of the proximal plunger rod 230p, the distal plunger rod 230d, and the first plunger rod 230a in FIG. 3E and in FIG. 3E. Distal advancement of the distal end 233b of the second plunger rod 230b a shorter extent than the distal advancement of the distal end 233a of the first plunger rod 230a allows the second plunger rod 230b to purge material (e.g., a selected amount of the second component 204b and/or air) from the second chamber 202b into a purge reservoir without ejecting all of the second component 204b from the second chamber 202b, while the first plunger rod 230a ejects more (e.g., most if not all) of the first component 204a from the first chamber 202a.

As noted above, a retainer 290 may be coupled with respect to the barrel 220 to limit various movements of the plunger assembly 230 with respect to the barrel 220. When the plunger assembly 230 reaches the position with respect to the barrel 220 illustrated in FIG. 3F, a pair of locking fingers 234a, 234b on the distal plunger rod 230d move distally past respective locking shoulders 294a, 294b on the retainer 290. As may be appreciated with reference to the detail view in FIG. 3Fs, showing a left side view of the plunger assembly 230 and retainer 290 illustrated in FIG. 3F, the locking fingers 234a, 234b and the locking shoulders 294a, 294b may be configured (e.g., tapered) to allow the locking fingers 234a, 234b to move distally past the locking shoulders 294a, 294b. However, the relative configurations of the locking fingers 234a, 234b and the locking shoulders 294a, 294b may be such that once the locking fingers 234a, 234b have moved distally past the locking shoulders 294a, 294b, the locking shoulders 294a, 294b prevent proximal movement of the locking fingers 234a, 234b. Accordingly, once the distal plunger rod 230d is moved into the position illustrated in FIG. 3F, the distal plunger rod 230d remains locked in that position as the proximal plunger rod 230p is moved back proximally, and the locking fingers 236 engage with the proximal end 231d of the distal plunger rod 230d (such as illustrated in FIG. 5C). At such time, the retainer 290 is removed from the multi-chamber device 210, allowing both plungers rods 230d, 230p to move distally together.

After the first component 204a has been injected into the third chamber 102c, such as illustrated in FIG. 3F, the multi-chamber system 200 may be shaken, such as schematically illustrated in FIG. 3G, such as to facilitate mixing of the first component 204a and the third component 204c within the vial 240. For the sake of convenience, and without intent to limit, the combination of the first component 204a and the third component 204c is referenced herein as a precursor 204d.

The precursor 204d is aspirated into the first chamber 202a by retracting the plunger assembly 230 proximally, as illustrated in FIG. 3H. It will be appreciated that in some instances, the entire content of the vial 240 (the combined first component 202a and third component 202c) is not aspirated out of the vial. 240. In some embodiments, the second component 204b (within the second chamber 202b) may react with the precursor 204d if combined therewith (e.g., the second component 204b may be an accelerant for completing a reaction between the first component 204a and the second component 204b). As such, as described above with respect to first component 204a and the second component 204b, the precursor 204d and the second component 204b may be maintained separate, such as by fluid isolation of the first chamber 202a and the second chamber 202b, until the user is ready for intentional combination of such components (e.g., when ready for delivery to a patient). As the precursor 204d is withdrawn from the vial 240, it may be desirable to avoid creation of a vacuum within the vial 240. In some embodiments, the fluid exchange device 280 is configured to provide venting, such as described, for example, in the above-incorporated provisional patent application ______, [ATTORNEY DOCKET 2001.3128100], and the vial adaptor 250 in such embodiments may thus be considered a vented vial adaptor 250.

In accordance with various principles of the present disclosure, retraction of the plunger assembly 230 prepares the multi-chamber device 210 for delivery of the precursor 204d and the second component 204b. More particularly, retraction of the plunger assembly 230 to the position in FIG. 3H brings the proximal plunger rod 230p into engagement with the distal plunger rod 230d so that the proximal plunger rod 230p and the distal plunger rod 230d can move together distally.

In the example of an embodiment of a plunger assembly 230 illustrated in FIG. 3H and the cross-sectional view along line IIIHx-IIIHx of FIG. 3H as illustrated in FIG. 3Hx, the proximal plunger rod 230p is retracted proximally with respect to the distal plunger rod 230d until one or more locking fingers 236 defined along the proximal plunger rod 230p are extended proximal to the proximal end 231d of the distal plunger rod 230d. In such configuration, the proximal plunger rod 230p engages the distal plunger rod 230d to move the distal plunger rod 230d distally when the proximal plunger rod 230p and the first plunger rod 230a are moved distally to eject the components 202b, 202p from the multi-chamber device 210. However, the above-described engagement of the locking fingers 234a, 234b on the distal plunger rod 230d and the locking shoulders 294a, 294b on the retainer 290 prevent distal movement of the distal plunger rod 230d. As such, the retainer 290 prevents inadvertent ejection of materials from the multi-chamber device 210 until the appropriate time.

With the multi-chamber device 210 ready, as illustrated in FIG. 3H, to deliver the precursor 204d from the first chamber 202a and the second component 204b from the second chamber 202b, the vial 240 and the vial adaptor 250 may be withdrawn from the multi-chamber device 210, as illustrated in FIG. 3I. As noted above, the sleeve lock 260 may be configured to be rotatably mounted on the vial adaptor 250. In such example of an embodiment, rotation of the sleeve lock 260 with respect to the multi-chamber device 210 (and optionally with respect to the vial adaptor 250 as well) allows the engaging elements 224′, 224p′ of the multi-chamber device 210 to rotate and then move generally axially out of engagement with the proximal engaging elements 264p, 264p′ of the sleeve lock 260 to release the sleeve lock 260 and the vial adaptor 250 for separation from the multi-chamber device 210. It will be appreciated that such movement may be generally opposite the movements of the sleeve lock 260 and its proximal-engaging elements 264p, 264p′ relative to the sleeve-lock-engaging elements 224, 224′ of the multi-chamber device 210 to engage the sleeve lock 260 and the multi-chamber device 210 with each other, such as described above.

A multi-chamber device 210 such as illustrated in FIG. 1 and FIG. 2 is configured to be fluidly coupled with a separate injection system 300 configured to deliver the components therein to a patient. For instance, the multi-chamber device 210 may not be configured to deliver the material directly to a patient. Instead, the multi-chamber device 210 is fluidly coupled with an injection system 300 which is configured to deliver the material directly to a patient. For instance, in the example of an embodiment of systems of the combining and/or delivery system 100 illustrated in FIG. 4A, the injection system 300 includes a base 310 with a proximal end 311 defining a connector portion 320 configured to be fluidly coupled with the multi-chamber device 210. More particularly, the injection system connector portion 320 defines nozzle ports 322a, 322b configured to be fluidly coupled with (e.g., seated with respect to, mated with, etc.) the nozzles 222a, 222b of the barrel 220 of the multi-chamber device 210. Once the nozzles 222a, 222b of the multi-chamber device 210 are fluidly coupled with (e.g., seated with respect to) the nozzle ports 322a, 322b of the injection system 300, the multi-chamber device 210 may eject components delivered therein (the second component 204b within the second chamber 202b, and the first component 204a or the precursor 204d within the first chamber 202a) via the injection system 300 into a patient. The multi-chamber device 210 and/or the injection system 300 may also combine components which are delivered separately by the multi-chamber device 210 to form the desired injectable material to be delivered to the patient.

In accordance with various principles of the present disclosure, a combining and/or delivery system 100 is configured to facilitate coupling of the multi-chamber device 210 with the injection system 300. For instance, to maintain fluid communication between the multi-chamber device 210 and the injection system 300 (e.g., to maintain fluid communication of the nozzles 222a, 222b and nozzle ports 322a, 322b), the injection system connector portion 320 includes one or more (e.g., 1-4, or more) engaging elements 324′ configured to engage with one or more injection-device-engaging elements 224i′ extending distally from the distal end 213 of the multi-chamber device 210 (e.g., from the distal end 223 of the barrel 220). More particularly, in the example of an embodiment illustrated in FIG. 4A (and also in FIG. 2), the injection-device-engaging elements 224i′ of the multi-chamber device 210 extend generally axially from the multi-chamber device 210 and have distal ends which are widened (in a direction transverse to the longitudinal axis LA as well as transverse to a radial direction with respect to the longitudinal axis LA) to form distal locking tabs 224 [symbol] ′. The injection-device-engaging elements 224i′ and their locking tabs 224 [symbol] ′ are sized, shaped, configured, and/or dimensioned to engage with the corresponding engaging elements 324′ and widened locking shoulders 324 [symbol] ′ defined in the base 310 of the injection system 300 (e.g., in the connector portion 320 thereof). In some embodiments, the sleeve-lock-engaging elements 224′ of the multi-chamber device 210 extend radially outwardly from the injection-device-engaging elements 224i′ and may widen at their radially outer ends into release tabs 228t′ to facilitate coupling of the injection-device-engaging elements 224i′ with the corresponding engaging elements 324′. As may be appreciated, because the locking tabs 224 [symbol] ′ are wider than the proximal ends of the engaging elements 324′ of the injection system 300, the locking tabs 224 [symbol] ′ cannot be extended distally into the proximal ends of the narrower proximal portions of the engaging elements 324′ of the injection system 300. In accordance with various principles of the present disclosure, the multi-chamber device 210 may be coupled with the injection system 300 by moving the multi-chamber device 210 and the injection system 300 axially together (along the directional arrows illustrated in FIG. 4A) and by biasing the locking tabs 224 [symbol] ′ radially-inwardly (such as by pressing radially-inwardly on the release tabs 228t′) so that the locking tabs 224 [symbol] ′ may fit into the entry openings 324e′ defined radially-inwardly of the engaging elements 324′ of the injection system 300 and along the proximal end 311 of the base 310 of the injection system 300 (e.g., along the proximal end of the connector portion 320). Once the locking tabs 224 [symbol] ′ are axially aligned with the corresponding locking shoulders 324 [symbol] ′ defined in the connector portion 320, the locking tabs 224 [symbol] ′ are allowed to extend radially outwardly to a neutral, unbiased locking position with respect to the locking shoulders 324 [symbol] ′, such as illustrated in FIG. 4B, and the detail FIG. 4Bd. In such position, the locking tabs 224 [symbol] ′ and locking shoulders 324 [symbol] ′ prevent axial separation of the multi-chamber device 210 and the injection system 300. Such configuration provides a relatively simple engagement and alignment of the multi-chamber device 210 and the injection system 300 for delivery of the contents of the multi-chamber device 210, via the injection system 300, to a patient. The multi-chamber device 210 may be separated from the injection system 300 (e.g., after ejecting the component 202b from within the second chamber 202b, and the first component 204a and/or the precursor 202d from within the first chamber 202a), by reversing the movements performed to couple the multi-chamber device 210 and the injection system 300. For instance, the release tabs 228t are pressed radially inwardly to disengage the locking tabs 224 [symbol] ′ from the locking shoulders 324 [symbol] ′ and the multi-chamber device 210 and injection system 300 may then be separated from each other.

In accordance with various principles of the present disclosure, one or more biasing elements 324 may be defined by the connector portion 320 of the base 310 of the injection system 300 to bias apart the multi-chamber device 210 and the injection system 300 axially to hold the locking tabs 224 [symbol] ′ in place with respect to the locking shoulders 324 [symbol] ′. For instance, one or more biasing elements 324 may be formed as a spring finger formed integrally with the connector portion 320 of the injection system base 310, and separated proximally therefrom by a slit. It may be appreciated that in some aspects, the configurations and relative positions of the biasing elements 324 and locking tabs 224 [symbol] ′ and nozzles 222a, 222b allows for “tilt” or “slop” of the assembly, transverse to the longitudinal axis LA. The biasing elements 324 may thus help to stabilize the assembly, as they extend transverse to the longitudinal axis LA. As may be appreciated with reference to FIG. 4B, the one or more biasing elements 324 may be positioned to engage the radially-outwardly extending sleeve-lock-engaging elements 224 of the multi-chamber device 210. Such formation of the biasing elements 324 and engagement with the sleeve-lock-engaging elements 224 may be facilitated by the different axial positions of the sleeve-lock-engaging elements 224, 224′ of the multi-chamber device 210 noted above. Optionally, seals (e.g., seals 326a, 326b, as illustrated in FIG. 6, and such as the above-described seals 226) may be provided to seal the connection between the multi-chamber device 210 and the injection system 300 and/or to provide a biasing force to hold the multi-chamber device 210 and the injection system 300 in engagement to resist inadvertent rotation therebetween, such as in a manner known to those of ordinary skill in the art.

Once the multi-chamber device 210 is securely coupled with the injection system 300, as illustrated in FIG. 6B, the chambers 202a, 202b of the multi-chamber device 210 are fluidly coupled with the injection system 300 to combine the second component 204b and the precursor 204d (or a first component 204a if not mixed with a third component 204c to form a precursor 204d) for delivery to a patient. The retainer 290 may be removed from the multi-chamber device 210 to allow the plunger assembly 230 to be advanced distally, such as illustrated in FIG. 4C, to eject the materials within the multi-chamber device 210 through the connector portion 320 and the support portion 330 of the injection system base 310. The materials are ejected through the injection system base 310 distally through a material delivery device 340 supported by the support portion 330 of the base 310. In some embodiments, the support portion 330 may be configured as/with a Y-connector to fluidly couple the two nozzle ports 322a, 322b defined in the connector portion 320 with the single lumen of the material delivery device 340, such as described in further detail in co-pending provisional patent application ______, titled DEVICES, SYSTEMS, AND METHODS FOR COMBINING AND/OR DELIVERING INJECTABLE MATERIALS, and filed on even date herewith [ATTORNEY DOCKET 2001.3127100], which application also is hereby incorporated by reference herein in its entirety and for all purposes. Optionally, the components which are fluidly isolated from each other within the multi-chamber device 210 are mixed within the injection system 300, such as within a mixing device therein (e.g., within the material delivery device 340, such as described in above-incorporated provisional patent application ______, [ATTORNEY DOCKET 2001.3125100].

As noted above, the injection system 300 may be configured not just to be coupled with the multi-chamber device 210, but may also be configured to be coupled with one or more other material-containing devices. For instance, as described above, in some instances, it may be desirable to administer a pre-treatment material to a patient before delivering an injectable material from the multi-chamber device 210 to the patient. In some aspects, a pre-treatment may be delivered by a pre-treatment system having a single fluid outlet (referenced herein as a nozzle for the sake of convenience and without intent to limit) configured for ejecting material into a patient via the injection system 300. In such instances, a combining and/or delivery system 100 formed in accordance with various principles of the present disclosure may include an adaptor system 400, such as illustrated in FIG. 1, and exploded in FIG. 5, configured to adapt an injection system 300 with two ports 322a, 322b to be able to fluidly communicate with a pre-treatment system with a single nozzle. It will be appreciated that although the additional material-containing device to be coupled with an injection system 300 formed in accordance with various principles of the present disclosure is described herein as a pretreatment device delivering a pretreatment material to a patient via the injection system 300, the present disclosure need not be limited in this regard. Reference herein is made to a pretreatment device 500 simply for the sake of convenience, and without intent to limit.

An example of an embodiment of an adaptor system 400 configured to facilitate coupling of an optional single-outlet pretreatment device 500 with an injection system 300 configured for coupling with a multi-chamber device 210 of the above-described combining and/or delivery system 100 is illustrated in FIG. 1, FIG. 5, and FIG. 6. The example of an embodiment of an adaptor system 400 formed in accordance with various principles of the present disclosure is illustrated in the exploded view of FIG. 5 as having an inlet portion 410 and an outlet portion 420. The illustrated example of an embodiment of an inlet portion 410 has a single port 412 extending proximally therefrom toward the proximal end 401 of the adaptor system 400. The illustrated example of an embodiment of an outlet portion 420 has a pair of nozzles 422a, 422b extending distally therefrom toward the distal end 413 of the adaptor system 400. It will be appreciated that the inlet portion 410 and the outlet portion 420 of an adaptor-system 400 formed in accordance with various principles of the present disclosure may be formed separately (such as illustrated), or as a single, monolithic element, the present disclosure not being limited in this regard. The single port 412 of the adaptor system 400 is configured to be fluidly coupled with a material transfer device 500 having a barrel 520 with a single nozzle 522, such as illustrated in FIGS. 7A-7D. The pair of nozzles 422a, 422b of the adaptor system 400 are configured to be fluidly coupled, respectively, with the injection system nozzle ports 322a, 322b, as may be appreciated with reference to FIG. 6.

The illustrated example of an embodiment of an adaptor system 400 includes injection-system-engaging elements 424i′ and locking tabs 424 [symbol] ′ and release tabs 428t configured to facilitate coupling of the adaptor system 400 with the injection system 300. In the illustrated example of an embodiment, the injection-system-engaging elements 424i′ and locking tabs 424 [symbol] ′ are provided (e.g., formed integrally or coupled with respect to) the outlet portion 420 of the adaptor-system 400, however other configurations are within the scope and spirit of the present disclosure, the present disclosure not being limited in this regard. The engagement of the injection system 300 and the adaptor-system 400 may be similar to the engagement of the injection system 300 with the multi-chamber device 210 so that the injection system 300 may utilize the same sleeve-lock-engaging elements 324′ and widened locking shoulders 324 [symbol] ′ for both the multi-chamber device 210 as well as the adaptor system 400. Reference accordingly is made to the above detailed description of the engagement of the multi-chamber device 210 and the injection system 300 as applicable, mutatis mutandis, to the engagement of the adaptor system 400 with the injection system 300. Briefly, the release tabs 428t may be pressed radially inwardly to insert the injection-system-engaging elements 424i′ with respect to the entry openings 324e′ defined radially-inwardly of the engaging elements 324′ of the injection system 300, and are released when the locking tabs 224 [symbol] ′ are axially aligned with the locking shoulders 324 [symbol] ′ to engage these elements to hold the adaptor system 400 and the injection system 300 from separating from each other.

Once the adaptor system 400 has been coupled with the injection system 300, the nozzle 522 of the pretreatment device 500 may be aligned with the port 412 of the adaptor system 400, as illustrated in FIG. 7A. The pretreatment device nozzle 522 may then be moved into engagement with the adaptor-system port 412, as illustrated in FIG. 7B, to fluidly couple the pretreatment device 500 with the injection system 300 with the assistance of the adaptor system 400. The plunger 530 of the pretreatment device 500 may then be advanced into the chamber 502 of the pretreatment device 500, as illustrated in FIG. 7CC, to expel pretreatment material 504 out from the chamber 502 for delivery to a patient via the adaptor system 400 and the injection system 300. The adaptor system 400 may be separated from the injection system 300 (e.g., after delivery of pretreatment material 504) by reversing the movements performed to couple the pretreatment device 500 and the injection system 300. For instance, the release tabs 428t are pressed radially inwardly to disengage the locking tabs 424 [symbol] ′ from the locking shoulders 324 [symbol] ′ and the adaptor system 400 and injection system 300 may then be separated from each other. It will be appreciated that the pretreatment device 500 may be separated/disengaged from the adaptor system 400 before the adaptor system 400 is separated/disengaged from the injection system 300. Optionally, the pretreatment device 500 may remain coupled with the adaptor system 400 to be separated/disengaged from the injection system 300 together with the adaptor system 400 (e.g., separated from the injection system 300 at the same time the adaptor system 400 is separated from the injection system 300, such as illustrated in FIG. 7D). The injection system 300 may be left in place (with the material delivery device 340 inserted in the patient) for coupling with a multi-chamber device 210 such as described above.

In view of the above, it will be appreciated that, in contrast with prior systems having multiple components which need to be delivered, assembled with respect to one another, separated from one another, manipulated together with and separately from one another, etc., the present disclosure simplifies devices, systems, and methods for combining and/or delivering an injectable material to a patient and/or reduces (if not eliminates) human error occurring with the use of complex prior systems. Moreover, it will be appreciated that the above-described configuration of engagement elements provides a relatively simple engagement and alignment of a multi-chamber device and a vial adaptor for combination of first component and a third component within the vial. Finally, it will be appreciated that the configuration of engagement elements may facilitate interchangeable engagement of the multi-chamber device with a vial adaptor or an injection system, and/or interchangeable engagement of the injection system with the multi-chamber device or an adaptor system.

Further in view of the above, it should be understood that the various embodiments illustrated in the figures have several separate and independent features, which each, at least alone, has unique benefits which are desirable for, yet not critical to, the presently disclosed devices, systems, and methods. Therefore, the various separate features described herein need not all be present in order to achieve at least some of the desired characteristics and/or benefits described herein. For instance, only one of the various features described above may be present in a device or system formed in accordance with various principles of the present disclosure. Alternatively, one or more of the features described with reference to one embodiment can be combined with one or more of the features of any of the other embodiments provided herein. That is, any of the features described herein can be mixed and matched to create hybrid designs, and such hybrid designs are within the scope of the present disclosure.

It is to be understood by one of ordinary skill in the art that the present discussion is a description of illustrative examples of embodiments only, and is not intended as limiting the broader aspects of the present disclosure. All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims. Various further benefits of the various aspects, features, components, and structures of devices, systems, and methods such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc.

Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A multi-chamber device comprising:
barrel defining a first chamber and a second chamber fluidly isolated from each other; and
a plunger assembly having a first plunger rod with a distal end extendable with respect to said first chamber, and a second plunger rod with a distal end extendable with respect to said second chamber;
wherein:
a portion of said first plunger rod and a portion of said second plunger rod move together with respect to said barrel; and
a portion of said first plunger rod is independently movable with respect to a portion of said second plunger rod to advance said distal end of first plunger rod with respect to said barrel a first extent and to advance said distal end of said second plunger rod with respect to said barrel a second extent different from said first extent.

2. The multi-chamber device of claim 1, wherein said portion of said first plunger rod and said portion of said second plunger rod which are independently movable are said distal end of said first plunger rod and said distal end of said second plunger rod.

3. The multi-chamber device of claim 2, wherein a portion of said first plunger rod is coupled with a portion of said second plunger rod so that said portion of said first plunger rod and said portion of said second plunger rod move together.

4. The multi-chamber device of claim 3, wherein said portion of said first plunger rod and said portion of said second plunger rod which move together are a proximal end of said first plunger rod and a proximal end of said second plunger rod.

5. The multi-chamber device of claim 4, wherein:
said second plunger rod comprises a proximal plunger rod and a distal plunger rod axially movable with respect to each other; and
said proximal plunger rod and said first plunger rod are coupled together to move together.

6. The multi-chamber device of claim 5, wherein said proximal plunger rod and said distal plunger rod are selectively movable into a position in which said proximal plunger rod and said distal plunger rod are engaged to move together.

7. The multi-chamber device of claim 6, further comprising a retainer coupled to said barrel to retain a portion of said second plunger rod in a positioned engaged with said retainer.

8. The multi-chamber device of claim 7, wherein said retainer engages said distal plunger rod in a distally advanced position with respect to said second chamber of said barrel.

9. The multi-chamber device of claim 8, wherein said proximal plunger rod and said distal plunger rod are selectively movable into a position in which said proximal plunger rod and said distal plunger rod are engaged to move together upon proximally retracting said proximal plunger rod with respect to said distal plunger rod.

10. The multi-chamber device of claim 1, wherein:
said second plunger rod comprises a proximal plunger rod and a distal plunger rod axially movable with respect to each other; and
said proximal plunger rod and said first plunger rod are coupled together to move together.

11. The multi-chamber device of claim 10, wherein said proximal plunger rod and said distal plunger rod are selectively movable into a position in which said proximal plunger rod and said distal plunger rod are engaged to move together.

12. The multi-chamber device of claim 1, further comprising a retainer coupled to said barrel to retain a portion of said second plunger rod in a positioned engaged with said retainer.

13. A multi-chamber system comprising:
a multi-chamber device comprising:
a barrel defining a first chamber and a second chamber fluidly isolated from each other; and
a plunger assembly having a first plunger rod with a distal end extendable with respect to said first chamber, and a second plunger rod with a distal end extendable with respect to said chamber;
wherein a portion of said first plunger rod is movable independently of a portion of said second plunger rod to advance said distal end of first plunger rod with respect to said barrel a first extent and to advance said distal end of said second plunger rod with respect to said barrel a second extent different from said first extent; and
a vial adaptor configured to be releasably coupled with said multi-chamber device to fluidly couple a separate chamber with said first chamber of said barrel.

14. The multi-chamber system of claim 13, wherein:
said second plunger rod comprises a proximal plunger rod and a distal plunger rod axially movable with respect to each other; and
said proximal plunger rod and said first plunger rod are coupled together to move together.

15. The multi-chamber system of claim 13, further comprising an injection system engageable with said multi-chamber device, wherein:
said multi-chamber device comprises one or more engaging elements;
said injection system comprises one or more engaging elements configured to engage with said one or more engaging elements of said multi-chamber device; and
said one or more engaging elements of said multi-chamber device are radially movable between an engaged position engaged with said one or more engaging elements of said injection system, and a disengaged position in which said multi-chamber device is axially movable with respect to said injection system.

16. The multi-chamber system of claim 15, further comprising an adaptor system, wherein said adaptor system comprises one or more engaging elements configured to engage with said one or more engaging elements of said injection system, and radially movable between an engaged position engaged with said one or more engaging elements of said injection system, and a disengaged position in which said multi-chamber device is axially movable with respect to said injection system.

17. A method of separately ejecting material from separate chambers within a multi-chamber device, said method comprising:
advancing a first plunger rod of the multi-chamber device with respect to a first chamber of the multi-chamber device a first extent; and
advancing a second plunger rod of the multi-chamber device with respect to a second chamber of the multi-chamber device a second extent different from the first extent;
wherein a portion of the second plunger rod is coupled with the first plunger rod to advance with the first plunger rod.

18. The method of claim 17, wherein advancing the second plunger rod comprises advancing a proximal portion of the second plunger rod with respect to a distal portion of the second plunger rod.

19. The method of claim 18, further comprising retracting the first plunger rod and the second plunger rod proximally with respect to the multi-chamber device barrel to engage the proximal portion of the second plunger rod with the distal portion of the second plunger rod to be advanceable together.

20. The method of claim 17, wherein:
advancing the first plunger rod comprises advancing a distal end of the first plunger rod with respect to the barrel a first distance; and
advancing the second plunger rod comprises advancing a distal end of the second plunger rod with respect to the barrel a second distance less than the first distance.
